(19) United States
(12) Patent Application Publication
(10) Pub. No.: US 2024/0209302 A1
(43) Pub. Date: Jun. 27, 2024

(54) SYSTEMS, METHODS, AND DEVICES FOR EX VIVO ANALYSIS OF RESECTED TISSUE SAMPLES

(71) Applicant: The United States of America,as represented by the Secretary,Department of Health and Human Services, Bethesda, MD (US)

(72) Inventors: Jonathan Matthew Hernandez, Bethesda, MD (US); Thomas J. Pohida, Bethesda, MD (US); Marcial Antonio Garmendia, Bethesda, MD (US); Samantha Marilyn Ruff, Bethesda, MD (US); Michael Martin Wach, Bethesda, MD (US); Shreya Gupta, Bethesda, MD (US); James McDonald, Bethesda, MD (US); Kirsten Remmert, Bethesda, MD (US); Alexander Joseph Rossi, Bethesda, MD (US)

(73) Assignee: The United States of America,as represented by the Secretary,Department of Health and Human Services, Bethesda, MD (US)

(21) Appl. No.: 17/910,911

(22) PCT Filed: Mar. 9, 2021
(86) PCT No.: PCT/US2021/021525

§ 371 Date: Feb. 15, 2023

Related U.S. Application Data
(60) Provisional application No. 62/988,783, filed on Mar. 12, 2020.

(51) Int. Cl. C12M 1/00; G01N 33/50

(52) U.S. Cl. CPC C12M 29/10; G01N 33/5082

(57) ABSTRACT

Ex vivo analysis can be performed by mounting a portion of live tissue resected from a patient on a sample platform. Using the sample platform, the resected tissue portion can be positioned within a perfusion chamber. Perfusate is flowed through the perfusion chamber and into contact with the resected tissue portion such that diffusion of oxygen occurs between the perfusate and the resected tissue portion. During the flowing, the resected tissue portion maintains a competent immune system. Drugs can be added to the perfusate flow to ascertain the effect on the tissue. The sample platform is designed to be removable from the perfusion chamber for analysis of the tissue by imaging or other investigation techniques, for experimental treatment, or for any other purpose. After removal, the sample platform can be returned to the perfusion chamber for continued viability of the tissue.

### CROSS-REFERENCE TO RELATED APPLICATION

This application claims the benefit of U.S. Provisional Application No. 62/988,783, entitled “Systems, Methods, and Devices for Ex Vivo Analysis of Resected Tissues Samples,” filed Mar. 12, 2020, which is incorporated by reference herein in its entirety.

### STATEMENT OF GOVERNMENT SUPPORT

This invention was made with Government support under project number ZIA BC 011759 by the National Institutes of Health (NIH). The Government has certain rights in the invention.

### FIELD

The present disclosure relates generally to evaluation of biological tissues, and more particularly, to ex vivo analysis of resected live tissue samples, such as primary or metastatic solid tumors.

### BACKGROUND

Potential treatment options for solid tumors continues to expand, but effective models to evaluate these new options prior to undertaking clinical trials remains limited. While mouse models have been developed in some cases, non-human models continue to be limited in terms of accuracy and application. Indeed, non-human models are non-existent for many cancers. Moreover, a greater understanding of the tumor microenvironment (TME), including dynamic interactions between cells in this heterogenous tissue, can help guide the development of new treatments as well as inform the prescription of existing treatment options. For example, immunotherapy has had been successful in the treatment of solid tumors, but some cancers are resistant to such treatments, with many individuals failing to respond to therapies that are otherwise effective for other similarly-diagnosed patients. Yet, such information about the fine-grained organization of cells and their functional state or interactions cannot be readily obtained by dispersed cell methods and standard sequencing techniques. Rather, a tumor model that accurately and faithfully recapitulates the heterogeneity of the human TME, inclusive of 3-D structure, stromal components, and immune populations is needed.

While patient-derived xenografts (PDX), patient-derived tumor organoids (PDO), and organ-on-chips have presented some progress over standard 2-D cell culture in modeling these complex interactions, they fail to provide the necessary prerequisites outlined above. For example, PDXs are cancer models where tissue resected from a human patient is implanted into a nonhuman, such as a mouse. As such, PDXs have stroma comprised of murine cells, lack a competent immune system, and represent a departure from human cell signaling and interactions. PDOs are generated by plating resected human tumor tissue in a basement membrane extract with a growth medium, but this model does not contain immune cells, blood vessels, or the necessary stromal cells. In organ-on-chips, different cell types can be added to the same model to observe interactions between tumor and healthy cells. However, cells from multiple patients are typically used to create the microenvironment, and the quantities of cells in the chip may not match normal physiological proportions. These models can suffer from genomic instability and evolution, and otherwise fail to imitate the tumor and its microenvironment with physiologic ratios and orientation of stromal and immune cells.

Accordingly, a need exists for a tumor model that accurately reflects the TME and can be used to test or customize treatment options. A further need exists for a model that is readily amenable to interrogation, such that resistance can be understood and rational combinations of treatment strategies can be ascertained. Indeed, mechanistic interrogation will move past the unrealistic “black and white” readouts currently available and allow understanding of how to best combine therapies for a desired effect. Embodiments of the disclosed subject matter may address one or more of the above-noted problems and disadvantages, among other things.

### SUMMARY

Embodiments of the disclosed subject matter provide systems, methods, and devices for ex vivo analysis of resected tissue samples, which can be used to provide an improved tumor model. A thin portion of tissue (e.g., a surface portion of the mesothelium) resected from a patient is mounted on a sample platform, which supports the tissue in a flow of perfusate within a perfusion chamber to allow exchange of nutrients, oxygen, water, and/or other chemicals (e.g., drugs or hormones) between the tissue and perfusate. Drugs can be added to the perfusate flow, for example, via an infusion port or valve within a fluid circuit connected to the perfusion chamber. The sample platform is designed to be removable from the perfusion chamber, for example, for analysis of the tissue (e.g., by imaging or other investigation technique), for treatment (e.g., partial or full immersion in a chemotherapeutic agent), or for any other purpose. After removal, the sample platform can be returned to the perfusion chamber for continued viability of the tissue ex vivo.

In some embodiments, when imaging with a microscope, the sample platform can be installed on an imaging platform holder, which holds the tissue on a viewing stage of the microscope and can easily reposition the tissue (e.g., by moving the tissue up or down) with respect to focal point of the microscope. In some embodiments, the sample platform can be installed on an exposure platform holder, which positions the tissue (or a portion thereof) within a fluid (e.g., stain for imaging, drug for treatment, etc.). In some embodiments, even though the sample platform is removed from the perfusion chamber, the sample platform, the imaging platform holder, and/or the exposure platform holder can include features that supply the tissue with perfusate and/or oxygen in order to maintain tissue viability outside the perfusion chamber.

In one or more embodiments, an ex vivo tissue analysis method comprises mounting a portion of live tissue resected from a patient on a sample platform. The method further comprises, using the sample platform, positioning the resected tissue portion within a perfusion chamber. The method also comprises flowing perfusate through the perfusion chamber and into contact with the resected tissue portion such that diffusion of oxygen occurs between the perfusate and the resected tissue portion. During the flowing, the resected tissue portion maintains a competent immune system.

In one or more embodiments, a system for ex vivo tissue analysis comprises a perfusion chamber and a sample platform. The perfusion chamber has an inlet, an outlet, and internal volume between the inlet and outlet. The sample platform has a tissue mount section and a chamber mount section coupled to the tissue mount section. The tissue mount section is constructed for mounting of a resected tissue portion thereon. The chamber mount section is constructed to releasably support the sample platform with respect to the perfusion chamber such that the resected tissue portion is positioned within the internal volume of the perfusion chamber. The tissue mount section of the sample platform has an opening that exposes a backside of the mounted resected tissue portion, such that both a frontside and a backside of the mounted resected tissue portion are exposed to perfusate within the perfusion chamber.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIG. 1A is a simplified schematic diagram illustrating aspects of a system for ex vivo tissue analysis, according to one or more embodiments of the disclosed subject matter.

FIG. 1B is a simplified schematic diagram illustrating aspects of a sample platform for use in the system, according to one or more embodiments of the disclosed subject matter.

FIG. 1C is a simplified schematic diagram illustrating alignment and locking features of the sample platform, according to one or more embodiments of the disclosed subject matter.

FIG. 1D is a simplified schematic diagram illustrating aspects of an imaging holder for use in positioning the sample platform for imaging, according to one or more embodiments of the disclosed subject matter.

FIG. 1E is a simplified schematic diagram illustrating operation of an exemplary ring gasket with an exemplary sample platform to provide an oxygenated microenvironment outside of the perfusion chamber, according to one or more embodiments of the disclosed subject matter.

FIG. 1F is a simplified schematic diagram illustrating an exemplary setup for exposing tissue to an experimental treatment outside of the perfusion chamber, according to one or more embodiments of the disclosed subject matter.

FIG. 1G is a simplified schematic diagram illustrating an exemplary setup for post-perfusion processing of the tissue, according to one or more embodiments of the disclosed subject matter.

FIG. 1H is a simplified schematic diagram depicting a generalized example of a computing environment in which the disclosed technologies may be implemented.

FIG. 2 is a process flow diagram of an exemplary method for ex vivo tissue analysis, according to one or more embodiments of the disclosed subject matter.

FIGS. 3A-3B are side and isometric views, respectively, of an exemplary sample platform, according to one or more embodiments of the disclosed subject matter.

FIG. 4A is an isometric view of an exemplary mounting jig that can be used to mount tissue on a sample platform, according to one or more embodiments of the disclosed subject matter.

FIGS. 4B-4C show perspective and side views, respectively, of the mounting jig of FIG. 4A in use to secure the sample platform of FIGS. 3A-3B.

FIGS. 5A-5B are side and isometric views, respectively, of an exemplary applicator that can be used to secure tissue on a sample platform, according to one or more embodiments of the disclosed subject matter.

FIGS. 5C-5D are side and isometric views, respectively, of the applicator of FIGS. 5A-5B in use to secure tissue on the sample platform of FIGS. 3A-3B.

FIGS. 6A-6C are exploded perspective, assembled perspective, and assembled side views, respectively, of an exemplary perfusion chamber, according to one or more embodiments of the disclosed subject matter.

FIGS. 6D-6E are perspective and side views, respectively, of the perfusion chamber of FIGS. 6A-6C with the sample platform of FIGS. 3A-3B.

FIG. 7A is a photograph of a fabricated example of a multi-station system for ex vivo tissue analysis.

FIG. 7B is a photograph of another fabricated example of a system for ex vivo tissue analysis having a reduced total perfusate volume.

FIG. 8A is an exploded isometric view of an exemplary imaging platform holder that can be used to position a sample platform for imaging, according to one or more embodiments of the disclosed subject matter.

FIG. 8B is a side view of a first member of the imaging platform holder of FIG. 8A.

FIGS. 8C-8D are side and isometric views, respectively, of the imaging platform holder of FIGS. 8A-8B in use to position the sample platform of FIGS. 3A-3B.

FIG. 9A show images of hematoxylin and eosin (H&E), immunohistochemical CD3, and immunohistochemical CD68 stains for peritoneum, liver capsule, and pleura tissue samples prior to (Day 0) and after four days (Day 4) in the system of FIG. 7A.

FIG. 9B shows images of H&E, immunohistochemical CD3, immunohistochemical CD68, and immunohistochemical CD20 stains for resected human peritoneum tissue samples prior to (Day 0) and after four days (Day 4) in the system of FIG. 7A.

FIGS. 9C-9D are graphs of Interferon-γ (IFN-γ) and interleukin 12 (IL-12) secretions, respectively, for the samples of FIG. 9A after four days in culture.

FIG. 10A shows images of H&E, CD3, CD68, and antigen Ki-67 stains for various cancer samples prior to being (Day 0) and after four days (Day 4) in the system of FIG. 7A.

FIG. 10B illustrates conservation of transcriptomics in hypoxic, oncogene/tumor suppressor, cancer stem cell, metabolism and epithelial to mesenchymal transition (EMT) panels over breast, gastric, pancreatic, and mesothelioma histology for the cancerous tumor samples of FIG. 10A.

FIG. 11A are various two-photon live images obtained of cancerous biopsies illustrating preservation of the metastatic microenvironment when in the system of FIG. 7A.

FIGS. 11B-11C are graphs of Interferon-γ (IFN-γ) and interleukin 12 (IL-12) secretions, respectively, for the samples of FIG. 11A after four days in culture.

FIG. 11D shows images of gastric cancer metastases to the peritoneum of FIG. 11A without (control) and with (IL-2) exposure to interleukin-2 (IL-2), illustrating an increase in velocity and displacement of CD45-positive cells.

FIGS. 11E-11F are graphs of velocity and displacement, respectively, for the cells of FIG. 11D.

FIG. 12A shows a two-photon image (top panel) obtained for a sidewall peritoneum sample from a gastric cancer patient that was subjected to a treatment using the system of FIG. 7A, and a timeseries montage of images (bottom panel) obtained for a liver capsule tissue sample from a colorectal cancer patient that was subjected to a treatment using the system of FIG. 7A.

FIG. 12B shows images of H&E, Ki67, succinate dehydrogenase (SDHD), 5-hydroxymethylcytosine (5-HMC), and 5-methylcytosine (5-MC) stains comparing a gastrointestinal stromal tumor (GIST) subjected to treatment using the system of FIG. 7A with a control sample.

FIG. 12C shows images of H&E and Ki67 stains comparing a resected cholangiocarcinoma tumor subjected to treatment using the system of FIG. 7A with a control sample.

FIG. 12D is a graph of the viable, degenerate, and necrotic cells for the tumor samples of FIG. 12C.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein, but are not intended to be limiting. For example, certain terms may be used such as “inside,” “outside,”, “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the appended claims.

### INTRODUCTION

Disclosed herein are exemplary systems, methods, and devices for ex vivo analysis of resected tissue samples, which can be used to provide an improved human tumor model. A thin portion of tissue (e.g., a surface portion of the mesothelium) can be resected from a patient and mounted on a sample platform. The sample platform can then be installed in a perfusion chamber and can support the tissue portion in a flow of perfusate to allow exchange of nutrients, oxygen, waste, water, and/or other substances (e.g., drugs or hormones). Drugs can be added to the perfusate flow, for example, via an infusion port or valve within a fluid circuit connected to the perfusion chamber.

The sample platform can be removed from the perfusion chamber for example, for analysis of the tissue (e.g., by microscopic imaging or other investigation technique), for experimental treatment (e.g., partial or full immersion in a chemotherapeutic agent), or for any other purpose. In some embodiments, when imaging with a microscope, the sample platform can be installed on an imaging platform holder, which holds the tissue on a viewing stage of the microscope and can easily reposition the tissue (e.g., by moving the tissue up or down with respect to the viewing stage), for example, to align different portions of the tissue with a focal point of the microscope. In some embodiments, the sample platform can be installed on an exposure platform holder, which positions the tissue (or a portion thereof) within a fluid (e.g., stain for imaging, drug for treatment, etc.). After removal, the sample platform can be returned to the perfusion chamber.

Unlike conventional tumor models, embodiments of the disclosed subject matter allow the tissue sample to maintain a competent immune system while preserving its tumor microenvironment (TME), which may offer more effective analysis of existing and new cancer treatment options and/or tailoring of a treatment to match the patient. Moreover, the configuration of the system (e.g., perfusion chamber, fluid circuit, sample platform, exposure platform holder, and/or imaging platform holder) can allow exposure of the tissue to treatment(s) (e.g., drugs, peripheral blood mononuclear cells (PBMCs), or other substances) while maintaining viability thereof. The system is also readily amenable to complex imaging, for example, to allow for analysis of a tumor's response to individual treatments on a cellular level.

### System Embodiments

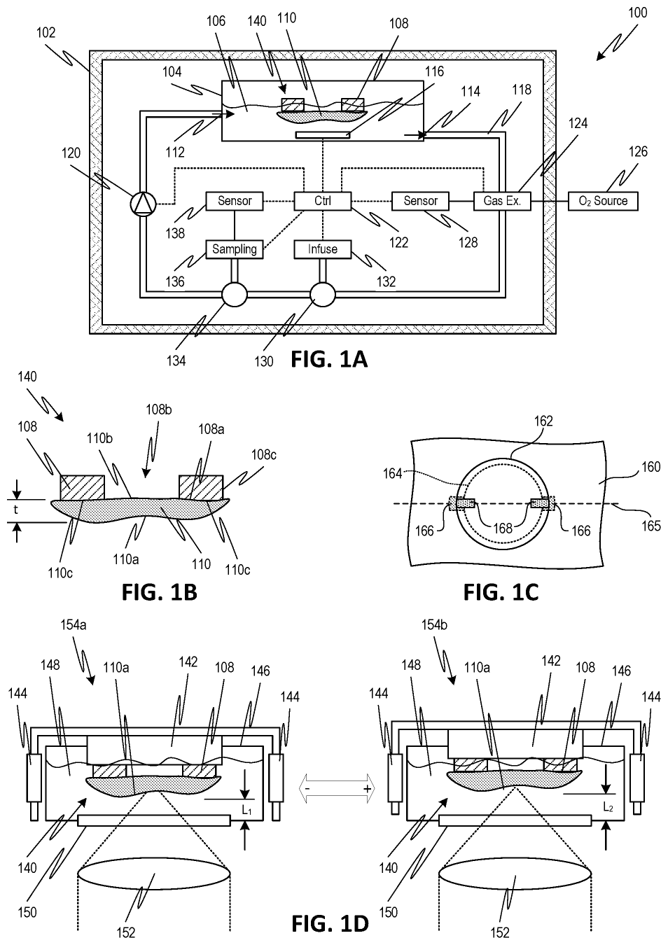

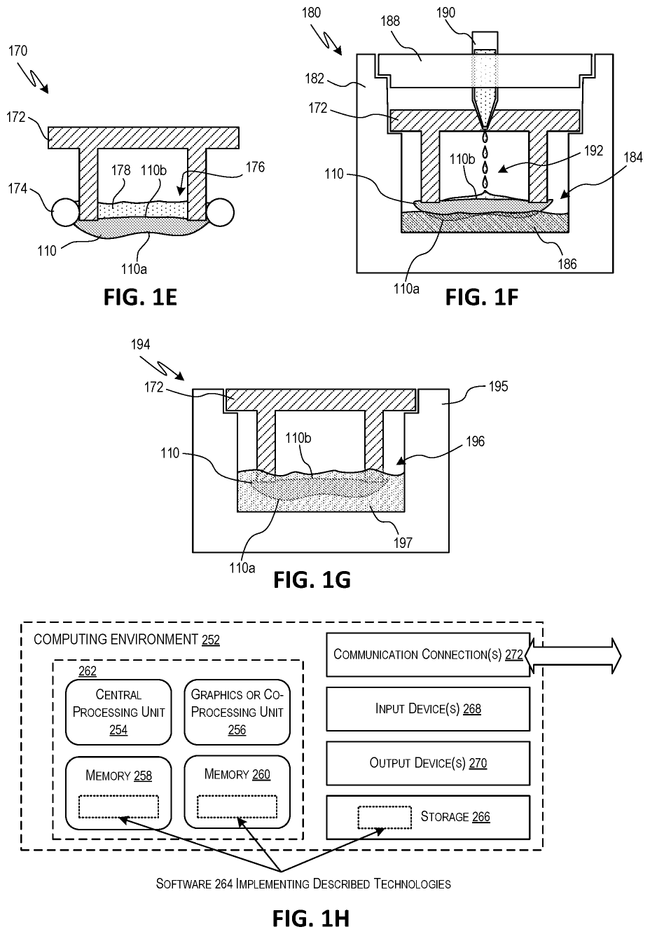

FIG. 1A illustrates a simplified schematic diagram of a generalized system 100 for ex vivo tissue analysis. The system 100 can include an environmental control apparatus 102, such as an incubator, which maintains an interior volume thereof at a predetermined temperature (e.g., 37° C.). A control system 122 (also referred to as a controller) may be provided within or external to the environmental control apparatus 102. The control system 122 can be coupled to the environmental control apparatus 102, as well as other components of system 100, for controlling operation thereof, either automatically or in response to commands from a user, for example, via a physical user interface (e.g., buttons, switches, knobs, etc.) or a graphical user interface (e.g., touch-screen display). Alternatively or additionally, each component can have its own separate control unit that operates independently of or in conjunction with control system 122. Alternatively, control system 122 can be omitted in favor of full manual operation by a user.

At least a perfusion chamber 104 can be disposed within the interior volume of the environmental control apparatus 102, although other components of system 100 may also be disposed therein. The perfusion chamber 104 has at least one inlet 112, through which perfusate can be introduced into an internal volume 106 thereof, and at least one outlet 114, through which perfusate can be extracted from the internal volume 106. The perfusate can comprise blood plasma or culture medium mixed with blood plasma. Optionally, the plasma (with or without culture medium) can be supplemented with one or more drugs, hormones, and/or nutrients (e.g., amino acids or amino acid precursors, glutathione, dextrose, antibiotics, and/or insulin). In some embodiments, the blood plasma is taken from the same patient as the tissue sample (e.g., autologous plasma).

In some embodiments, the inlet 112 may be misaligned with respect to the outlet 114, so as to encourage adequate mixing of perfusate within internal volume 106 of the perfusion chamber 104. For example, the inlet 112 may be at a height (as measured from a bottom of the perfusion chamber) greater than that of the outlet 114, or vice versa. In another example, one or both of the inlet 112 and outlet 114 can be offset from a radial direction of the perfusion chamber 104 in a plane perpendicular to the direction of gravity. The perfusion chamber 104 can optionally include additional passive structures or active structures designed to further agitate the perfusate within internal volume 106 of perfusion chamber 104 to encourage adequate mixing. Control system 122 can be operatively connected to the one or more active structures to effect control thereof. Exemplary active structures can include, but are not limited to, a magnetic stirrer bar 116 that spins within the internal volume 106 upon application of an externally applied magnetic field. Alternatively, in some embodiments, the perfusion chamber 104 is provided without any active structures for perfusate flow agitation. Exemplary passive structures can include, but are not limited to, baffles on bottom, side, or top internal surfaces of the internal volume 106, configuration or arrangement of inlet 112 or outlet 114, and orientation of perfusate flow (from inlet 112 to outlet 114) within the perfusion chamber. For example, inlet 112 can include a nozzle or other structure designed to instigate turbulent flow of perfusate within the internal volume 106 to enhance mixing. In another example, multiple inlets 112 and/or multiple outlets 114 can be arranged around a circumference of the perfusion chamber 104. Alternatively, in some embodiments, the perfusion chamber 104 is provided without any additional passive structures for perfusate flow agitation.

In some embodiments, an oxygen-generating biomaterial (OGB) can be disposed within, or at least in contact with, perfusate in the perfusion chamber. The OGB can be configured to release oxygen into the perfusate by diffusion of entrapped, absorbed oxygen or by chemical generation of oxygen. OGB materials can include, but are not limited to, sodium percarbonate (SPO), calcium peroxide (CaO₂), magnesium peroxide (MgO₂), and hydrogen peroxide (H₂O₂) (which may be combined with catalase or other antioxidant) contained in a supporting structure formed from hydrogel, ethyl cellulose, or a biocompatible polymeric material (e.g., polydimethylsiloxane (PDMS), poly(lactic-co-glycolic acid) (PLGA), etc.). For example, calcium peroxide powder can be mixed with PDMS and loaded into a cylindrical silicone mold (e.g., having a depth of 1 mm) for curing. After curing, the PDMS-CaO₂ disk can be removed from the mold and disposed at a bottom of the perfusion chamber 104 aligned with, or at least proximal to, sample 110.

The OGB structure may continuously release O₂ into the perfusate, which may supplement the oxygen infusion by the gas exchange unit 124, described below. Alternatively, the OGB can form a part of the perfusion chamber 104 (e.g., coating of a surface of the chamber), or be coupled to or integrated with other structures within the perfusion chamber (e.g., as part of stirrer bar 116 or sample platform 108). When multiple samples 110 are provided in the perfusion chamber 104, each sample may have its own separate OGB structure.

An external fluid circuit 118, composed of one or more fluid conduits (e.g., glass, metal, or polymer tubing), connects the inlet 112 and outlet 114 together so as to recirculate exiting perfusate back to the perfusion chamber 104. One or more pumps 120 (e.g., a peristaltic pump) is provided to move perfusate through the fluid circuit 118, and thereby cause flow of perfusate through internal volume 106 of the perfusion chamber 104. Alternatively, the external fluid circuit 118 may have a non-recirculation configuration, for example, where a first conduit network of the fluid circuit connects inlet 112 to a source of fresh perfusate and a second conduit network of the fluid circuit connects outlet 114 to a receptacle (e.g., for waste collection or recycling) or drain (e.g., for waste disposal). In such a configuration, each fluid conduit network may have a respective pump 120. Control system 122 can be operatively connected to the one or more pumps 120 to effect control thereof.

A gas exchange unit 124 (also referred to as a gas exchanger) can be connected to the external fluid circuit 118. The gas exchange unit 124 can be configured to infuse perfusate flowing therethrough with gaseous oxygen from a source 126 (in addition to or in place of OGB structures) and to remove gaseous carbon dioxide from the perfusate. For example, source 126 can be an oxygen gas canister or oxygen supply line, either of which may be located external to the environmental control apparatus 102. In some embodiments, source 126 provides humidified oxygen to help compensate for any fluid loss during the flowing of perfusate. A sensor 128 can monitor the perfusate flowing through the gas exchange unit 124 (or monitor perfusate within internal volume 106) to provide feedback for regulation of gas exchange, for example, by sending a signal to control system 122. In some embodiments, the gas exchange unit 124 can comprise an oxygenator, which has a perfusate flow path therein separated from an air flow path therein by a membrane. For example, the oxygenator can have a size of 1000 cm² or less, preferably 100 cm² or less. The inlet end of the oxygenator air flow path can be connected to the source of oxygen while the outlet end of the oxygenator air flow path can be connected to an outlet valve. The control system 122 can be operatively coupled to the outlet valve to effect control thereof, for example, to cause venting of CO₂ gas (e.g., external to the environmental control apparatus 102) to achieve a predetermined pH as measured by sensor 128. Alternatively or additionally, gas exchange unit 124 can comprise a gas mixer that blends together gases from an oxygen source (e.g., source 126) and a CO₂ source (not shown) to supply a dynamic, customized gas mixture to the perfusate. For example, the control system 122 can control the gas mixer to change the gas mixture composition in order to manipulate the levels of CO₂ dissolved in the perfusate and thereby adjust the pH of the perfusate to be within a predetermined range or to maintain the pH at a predetermined value.

An infusion device 132 can be connected to the external fluid circuit 118 by way of connection 130. For example, connection 130 can comprise a valve (e.g., a multi-position valve), a union (e.g., a Y-junction), an injection or infusion port, or any other fluidic structure that allows the combination of the infused substance and the perfusate (or a portion thereof) in fluid circuit 118. The infusion device 132 can be used to continuously or periodically inject a substance (e.g., drug, hormone, fluid, cells such as PBMCs, or any other chemical or substance) into the flowing perfusate. For example, the infusion device 132 can comprise a syringe pump or an infusion pump. In some embodiments, the infusion device 132 can continuously (substantially continuous while perfusate is flowing, except for brief periods of downtime for setup, replacement, or repair) provide insulin and/or sterilized water to the fluid circuit. Alternatively or additionally, the infusion device 132 can provide a single injection or provide periodic injections of one or more drugs, for example, to test a particular treatment option for cancer, and/or cells, for example, PBMCs isolated from the patient. The control system 122 can be operatively coupled to the infusion device 132 to effect control thereof, for example, to cause control a rate and/or timing of infusion. The control system 122 also may be operatively coupled to the connection 130, for example, to configure a multi-position valve to allow infusion by infusion device 132.

In some embodiments, a sampling device 136 can also be connected to the external fluid circuit 118 by way of connection 134. For example, connection 134 can comprise a valve (e.g., a multi-position valve), a union (e.g., a Y-junction), a withdrawal port, or any other fluidic structure that allows removal of a portion of the perfusate in fluid circuit 118. The sampling device 136 can be used to continuously or periodically withdrawal a portion of the perfusate from fluid circuit 118, for example, for interrogation by sensor 138 or for testing outside of system 100. For example, perfusate may be sampled periodically by sampling device 136 to test physiological parameters such as pH, oxygen, and metabolite levels using sensor 138. Alternatively or additionally, the perfusate can be sampled continuously or periodically to ascertain biomarker levels therein, for example, to assess response of the tissue (or a tumor therein) to a drug in the perfusate or to previously performed treatment outside of the perfusion chamber. The control system 122 can use feedback from sensor 138 (e.g., via one or more signals) to control gas exchange unit 124, infusion device 132, or a perfusate supply (not shown) to take corrective action, or to notify a user to take corrective action.

A tissue sample 110 is positioned within the flowing perfusate in the internal volume 106 of the perfusion chamber 104 by a sample platform 108. The sample platform 108 can include one or more structures (not shown) that releasably couple the sample platform 108 to the perfusion chamber 104. For example, the sample platform 108 can include a tissue mount section to which the tissue sample 110 is mounted, a chamber mount section, and one or more arms connecting the tissue mount section to the chamber mount section. The chamber mount section can be configured to interact with a top, bottom, side, or any other portion of the perfusion chamber 104 or surrounding structures to hold the tissue mount section (and the tissue sample 110 mounted thereon) in position within the flowing perfusate within the internal volume 106 of the perfusion chamber 104.

In some embodiments, the chamber mount section of sample platform 108 and a portion of the perfusion chamber 104 can comprise one or more cooperating features that align and/or retain (e.g., lock) the sample platform in a predetermined orientation with respect to the perfusion chamber 104 and/or contents thereof. For example, as shown in FIG. 1C, a portion 160 of the perfusion chamber (or a structure otherwise coupled to the perfusion chamber) can have an opening 164 through which a tissue mount section of a sample platform is inserted to access the internal volume of the perfusion chamber. The chamber mount section 162 of the sample platform can have a diameter greater than that of opening 164. The chamber mount section 162 thus sits on an external surface of the perfusion chamber portion 160, thereby supporting the tissue mount section within the perfusion chamber. The chamber mount section 162 can have one or more protrusions 168 that fit within respective recesses 166 of the perfusion chamber portion 104a. Alternatively or additionally, the perfusion chamber portion 104a can have one or more protrusions that fit within respective recesses of chamber mount section 162.

The cooperating features of recesses and protrusions act to position the chamber mount section 162 and the attached tissue mount section in a fixed orientation with respect to the perfusion chamber, for example, with respect to a direction of perfusate flow in the perfusion chamber. Moreover, in some embodiments, the cooperating features can resist rotation of the sample platform, for example, due to forces of the perfusate flow on the tissue mount section or other portions of the sample platform. In some embodiments, the cooperating features can be keyed, such that only one orientation is possible for the sample platform installed to the perfusion chamber. For example, in some embodiments, the chamber mount section 162 can have a single protrusion 168, and the perfusion chamber portion 104a has a single recess 166. In another example, the chamber mount section 162 can instead have a pair of protrusions 168 that are not diametrically aligned (e.g., not disposed on a common axis 165 passing through a center of opening 164), and the perfusion chamber portion 104a can have a corresponding arrangement of recesses 166. Other configurations for such cooperating features to align and/or retain the sample platform are also possible according to one or more contemplated embodiments.

Referring to FIG. 1B, a close-up view of the tissue sample 110 mounted on the sample platform 108 to form tissue assembly 140 is shown. The tissue sample 110 may be a relatively thin (e.g., having a thickness, t, less than 1 mm) piece of live tissue that has been recently resected from a patient (e.g., less than 10 minutes, and preferably less than 2 minutes). The tissue sample 110 can have a front surface 110a (e.g., a natural external surface of the tissue, also referred to as a frontside) and a back surface 110b (e.g., a surface of the tissue exposed by the resection, also referred to as a backside). The sample platform 108 can have a mounting surface 108a over which the back surface 110b of the tissue sample 110 is disposed. However, the sample platform 108 includes an aperture 108b that exposes most, or at least a majority, of the back surface 110b. Thus, only the periphery 110c (or a peripheral portion) of the tissue sample 110 is in contact with and covered by the mounting surface 108a of the sample platform 108.

In some embodiments, the periphery 110c of the tissue sample 110 can extend beyond edges of the tissue mount section of the sample platform 108. In such configurations, the tissue sample 110 may overhang and come into contact with sides 108c and/or a surface of the tissue mount section opposite the mounting surface 108a. The overhanging portion of the tissue sample 110 may be used to secure the sample to the tissue mount section without otherwise preventing perfusate access or obscuring visual inspection of the front surface 110a. The tissue sample 110 can be mounted on the sample platform 108 using mechanical attachment (e.g., suture wrapped around the tissue overhang and side 108c, a rubber band placed around the tissue overhang and side 108c, etc.) or a medical or biocompatible adhesive (e.g., between periphery 110c and mounting surface 108a).

In operation, tissue assembly 140, formed by the mounting of tissue sample 110 on sample platform 108, is inserted into perfusion chamber 104 such that tissue sample 110 is fully submerged within the perfusate within the internal volume 106, as shown in FIG. 1A. Perfusate is pumped through fluid circuit 118 to inlet 112 and from outlet 114 via pump to induce a flow of perfusate within internal volume 106. Gas exchange unit 124 and/or OGB structure (not shown) infuse oxygen into the flowing perfusate. In some embodiments, gas exchange unit 124 can infuse CO₂ into or remove CO₂ from the perfusate in order to control a pH thereof. The relatively thin thickness of the tissue sample 110, coupled with the exposure to perfusate of both front surface 110a and back surface 110b of the sample 110 by the sample platform 108, allows nutrients, oxygen, and other substances (e.g., drugs, hormones, PBMCs, etc.) to diffuse from the perfusate to most (and preferably all) cells of the tissue sample 110 while waste and CO₂ diffuse from most (and preferably all) cells of the tissue sample 110 into the perfusate, thereby supporting the viability of the tissue sample 110 ex vivo. Moreover, since the native structure of the tissue sample 110 has not otherwise been compromised by the resection and mounting, the tissue sample 110 retains its original microenvironment (e.g., tumor microenvironment (TME) in the case of a cancerous section) including a competent immune system. That is, despite being removed from the patient, the tissue sample 110 maintains its native heterogenous composition of stromal components and 3-D microstructure, as well as immune populations capable of exhibiting an immune response, thereby providing an ex vivo tumor model that is more accurate than heretofore achievable.

When the tissue sample 110 is of a tumor (e.g., a primary cancerous tumor or a metastasis thereof), a drug under investigation for treatment of the tumor can be injected into the perfusate, for example, using infusion device 132 and fluid circuit connection 130. Alternatively, the tissue sample 110 may be of healthy tissue, and the drug administration may serve as an experimental control or to assess side effects. Again, the relatively thin thickness of the tissue sample 110, coupled with the exposure to perfusate of both front surface 110a and back surface 110b of the sample 110 by the sample platform 108, allows the drug to diffuse from the perfusate to most (and preferably all) cells of the tissue sample 110.

To investigate the effect of the drug on the tissue, the tissue assembly 140 can be removed from the perfusion chamber 104 for interrogation, for example, for microscopic imaging. Alternatively or additionally, the ability to interrogate the tissue sample 110 may be built into system 100, for example, by providing an objective lens of the microscope within the environmental control apparatus 102 at an appropriate location outside perfusion chamber 104, with a wall of the perfusion chamber 104 between the tissue sample 110 and the objective lens being transparent.

In some embodiments, the tissue assembly 140 can be removed from the perfusion chamber 104 and temporarily mounted to an imaging platform holder 142 for imaging of tissue sample 110 by a microscope (e.g., with objective lens 152), as shown in FIG. 1D. In particular, the tissue sample 110 can be held within fluid 148 (e.g., culture medium) in an imaging dish 146, which may have a bottom surface with a viewing port 150 (e.g., glass or other optically transparent material window). The imaging platform holder 142 can have one or more displacement members 144 that can be actuated to move the tissue sample 110 with respect to the viewing port 150, for example, between a first configuration 154a where front surface 110a of the tissue sample 110 is at a closest distance, L₁, to the viewing port 150 and a second configuration 154b where front surface 110a of the tissue sample 110 is at a farthest distance, L₂, from the viewing port 150. In some embodiments, the displacement members 144 of the imaging platform holder 142 can be controlled to position different portions of the tissue sample 110 with respect to a focal plane of the imaging apparatus (e.g., microscope). After imaging, the tissue assembly 140 can be removed from the imaging platform holder 142 and returned to the perfusion chamber 104.

In some embodiments, the tissue assembly can be adapted to maintain viability of the tissue sample over an extended period of time (e.g., ˜12 hours) outside of the perfusion chamber, for example, to allow for prolonged imaging, treatment, transport, or any other reason. For example, FIG. 1E illustrates a tissue assembly configuration 170 employing a ring gasket 174 disposed around a circumferential portion of sample platform 172 adjacent to the tissue mount section thereof. The ring gasket 174 thus defines a well region 176 at the back surface 110b of the tissue sample 110, which well region 176 can be filled with perfusate 178 (e.g., plasma and/or culture medium). The front surface 110a of the tissue sample 110 can be unobstructed by the ring gasket 174, and imaging and/or treatment can proceed without interference. In some embodiments, the ring gasket 174 can be formed of an oxygen-generating biopolymer (OGB), such as those OGB materials described above with respect to the perfusion chamber. The ring gasket 174 formed of OGB can release oxygen into the perfusate 178 within well 176, thereby providing the tissue sample 110 with an oxygenated microenvironment outside of the perfusion chamber that can sustain the tissue sample 110 until it can be returned to the perfusion chamber.

In some embodiments, the tissue sample 110 can be removed from the perfusion chamber and disposed in contact with a fluid or otherwise subjected to a treatment outside of the perfusion chamber. For example, in some embodiments, part or all of the tissue sample 110 can be immersed in a fluid (e.g., stain for imaging or a chemotherapeutic agent). In such embodiments, the sample platform can be installed within an exposure platform holder, which positions the tissue sample (mounted on the sample platform) at a predetermined position with respect to the fluid. In some embodiments, the exposure platform holder can be adapted to maintain viability of the tissue sample during the immersion and/or treatment (e.g., 60-90 minutes). For example, FIG. 1F illustrates a treatment setup 180 where sample platform 172 is disposed within an exposure platform holder 182 having a reservoir 184 filled with a fluid 186 (e.g., a drug, such as a chemotherapeutic agent). The exposure platform holder 182 can position the tissue sample 110 within the reservoir 184 such that the front surface 110a is immersed in the fluid 186 while the back surface 110b remains exposed. FIG. 1G illustrates an alternative setup 194 where sample platform 172 is disposed within an exposure platform holder 195 having a reservoir 196 filled with a fluid 197 (e.g., an imaging stain or a drug). The exposure platform holder 195 can position the tissue sample 110 within the reservoir 196 such that both the front surface 110a and the back surface 110b are immersed in fluid 197.

In some embodiments, the exposure platform holder can be adapted to maintain viability of the tissue sample 110 over a predetermined period (e.g., ˜60-90 minutes for a chemotherapeutic treatment regimen). For example, exposure platform holder 182 can include a fluid supply 190 supported by member 188 to deliver perfusate (e.g., plasma and/or culture media) to prevent the tissue sample 110 from drying out and/or to provide nutrients to the tissue sample 110. In some embodiments, the perfusate supplied to the tissue sample 110 can be oxygenated, for example, by dissolving oxygen in the perfusate within supply 190 or by providing an appropriate OGB structure within supply 190 or adjacent to tissue sample 110. The fluid supply 190 may be configured to deliver the perfusate to the back surface 110b of the tissue sample 110 as a substantially continuous or periodic supply of droplets 192.

Although the above discussion has focused on a single tissue sample 110 within a perfusion chamber 104 and a single perfusion chamber 104 within environmental control apparatus 102, embodiments of the disclosed subject matter are not limited thereto. Rather, any number of the above described components are possible according to one or more contemplated embodiments. Indeed, multiple tissue samples (with respective sample platforms) can be provided within a single perfusion chamber 104. For example, different tissue samples (e.g., healthy and cancerous tissue) can be provided in the same perfusion chamber 104 and subjected to the same investigation conditions (e.g., drug exposure, PBMCs, etc.) to compare efficacy and/or determine side effects. For example, in some embodiments, each perfusion chamber 104 can support four sample platforms 108 therein with respective tissue samples 110. In such embodiments, the four sample platforms 108 may be symmetrically arranged with respect to the perfusate flow through the perfusion chamber 104, for example, with two sample platforms 108 on opposite sides of a perfusate axis extending from the inlet 112 to the outlet 114.

Moreover, multiple perfusion chambers 104 (with respective fluid circuits 118) can be provided within environmental control apparatus 102 and may share one or more of the system 100 components, such as pump 120, control system 122, and/or oxygen source 126. For example, different perfusion chambers 104 can be provided to allow for parallel testing of multiple tissue samples. In some embodiments, two perfusion chambers 104 (e.g., each with four sample assemblies therein) can share a single pump 120. Other configurations are also possible according to one or more contemplated embodiments.

FIG. 1H depicts a generalized example of a suitable computing environment 252 in which the described innovations may be implemented, such as control system 122. The computing environment 252 is not intended to suggest any limitation as to scope of use or functionality, as the innovations may be implemented in diverse general-purpose or special-purpose computing systems. For example, the computing environment 252 can be any of a variety of computing devices (e.g., desktop computer, laptop computer, server computer, tablet computer, etc.). In some embodiments, the computing environment is an integral part of a tissue analysis system. Alternatively, in some embodiments, the computing environment is a separate system connected to the tissue analysis system, for example, by making operative electrical connections (e.g., wired or wireless) to the tissue analysis system or components thereof.

With reference to FIG. 1H, the computing environment 252 includes one or more processing units 254, 256 and memory 258, 260. In FIG. 1H, this basic configuration 262 is included within a dashed line. The processing units 254, 256 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 1H shows a central processing unit 254 as well as a graphics processing unit or co-processing unit 256. The tangible memory 258, 260 may be volatile memory (e.g., registers, cache, RAM), non-volatile memory (e.g., ROM, EEPROM, flash memory, etc.), or some combination of the two, accessible by the processing unit(s). The memory 258, 260 stores software 264 implementing one or more innovations described herein, in the form of computer-executable instructions suitable for execution by the processing unit(s).

A computing system may have additional features. For example, the computing environment 252 includes storage 266, one or more input devices 268, one or more output devices 270, and one or more communication connections 272. An interconnection mechanism (not shown) such as a bus, controller, or network interconnects the components of the computing environment 252. Typically, operating system software (not shown) provides an operating environment for other software executing in the computing environment 252, and coordinates activities of the components of the computing environment 252.

The tangible storage 266 may be removable or non-removable, and includes magnetic disks, magnetic tapes or cassettes, CD-ROMs, DVDs, or any other medium which can be used to store information in a non-transitory way, and which can be accessed within the computing environment 252. The storage 266 can store instructions for the software 264 implementing one or more innovations described herein.

The input device(s) 268 may be a touch input device such as a keyboard, mouse, pen, or trackball, a voice input device, a scanning device, or another device that provides input to the computing environment 252. The output device(s) 270 may be a display, printer, speaker, CD-writer, or another device that provides output from computing environment 252.

The communication connection(s) 272 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java, Perl, any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Method Embodiments

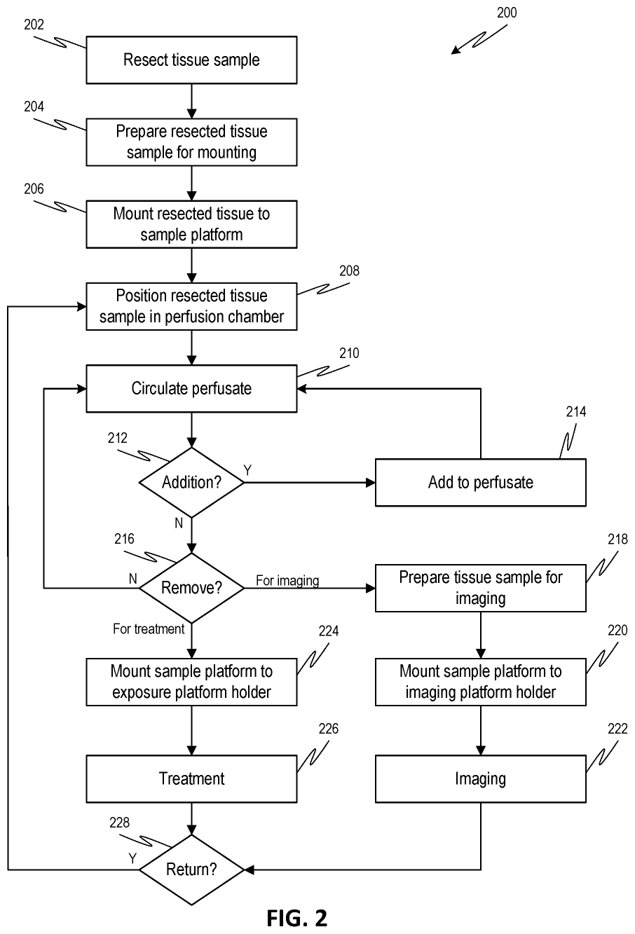

Referring to FIG. 2, an exemplary method 200 for ex vivo tissue analysis is shown. The method can begin at process block 202, where a tissue sample is resected from a patient. The resected sample may be of healthy tissue (e.g., to use as an experimental control or to test drug side effects), cancerous tissue, or a combination thereof. In some embodiments, the resected sample is of a solid tumor. For example, the resected sample can be of metastases to the mesothelium (e.g., peritoneum, pleura, liver capsule, etc.) or of normal mesothelium adjacent to a tumor-bearing mesothelium. The tissue resection may be performed relatively early after initiating a surgical procedure on the patient (e.g., within the first hour of surgery) so as to avoid neutrophil infiltrate to the sample that is otherwise reflective of the ongoing injury response to the surgery rather than in vivo biology.

The method 200 can proceed to process block 204, where the resected sample is prepared for mounting to the sample platform. The tissue resection may be transported from the surgical center to a preparation stage and disposed in a warmed bath of culture medium. For example, the preparation can include trimming a size of the resected sample (e.g., to fit a size or shape of the tissue mount section of the sample platform) and/or removing excess tissue (e.g., adipose tissue) from the sample. In embodiments, the prepared tissue sample may be relatively thin, for example, having a thickness less than 1 mm.

The method 200 can proceed to process block 206, where the resected sample is mounted to the sample platform, in particular, the tissue mount section thereof. The resected sample can be positioned over the tissue mount section with its native surface (e.g., mesothelial surface) facing away from the tissue mount section and such that regions of interest (e.g., a macroscopically visible tumor) are centered on the tissue mount section. A periphery of the sample can then be attached to the tissue mount section, for example, using a circumferential attachment member (e.g., a 1-0 or 0 silk suture, a rubber band, etc.). A time period between the resection of tissue (process block 202) and completion of the tissue mounting (process block 206) may be less than or equal to five minutes, and preferably less than two minutes.

The method 200 can proceed to process block 208, where the sample platform is inserted into the perfusion chamber, such that the mounted tissue sample is positioned within perfusate in the perfusion chamber. For example, the perfusion chamber can include one or more receptacles in a surface thereof that allow the tissue mount section to pass therethrough but prevent the chamber mount section of the sample platform from passing. The chamber mount section thus releasably supports the sample platform on the perfusion chamber, with the tissue sample being suspended within the perfusion chamber.

The method 200 can proceed to process block 210, where perfusate is flowed through the perfusion chamber and circulated from the perfusion chamber outlet back to the perfusion chamber inlet via an external fluid circuit. As noted above, the perfusate can comprise blood plasma (e.g., autologous plasma, obtained from the same patient as the tissue sample) or culture medium mixed with plasma. Optionally, the plasma can be supplemented with one or more drugs, hormones, and/or nutrients (e.g., amino acids or amino acid precursors, glutathione, dextrose, antibiotics, and/or insulin). The relatively-thin thickness of the tissue sample, coupled with the exposure to perfusate offered by the sample platform, allows nutrients and oxygen (e.g., supplied by a gas exchange unit, such as an oxygenator, a gas mixer, and/or an OGB structure within the perfusion chamber) to diffuse from the perfusate to cells of the tissue sample while waste and carbon dioxide diffuse from the cells into the perfusate, thereby supporting the viability of the tissue sample ex vivo.

The method 200 can proceed to decision block 212, where it is determined if the tissue sample should be exposed to a substance. For example, the drug could be a potential treatment option for a cancer of the patient. The tissue sample thus serves as a model to assess efficacy of the drug for the patient's cancer. The determination at decision block 212 can be based on timing (e.g., for periodic drug exposure or waiting a period after introduction of the sample to the perfusion chamber to allow it to equilibrate), a status of the tissue sample (e.g., whether the tissue sample is to serve as an experimental control), or any other criteria. If perfusate-based drug exposure is desired at decision block 212, the method 200 can proceed to process block 214, where a drug is added to the perfusate. The method 200 can then return to process block 210 for perfusate circulation, such that the drug is transported to the tissue sample in the perfusion chamber via the flowing perfusate for interaction with cells of the sample.

Alternatively or additionally, decision block 212 can include determining whether the tissue sample should be exposed to PBMCs. For example, PBMCs can be isolated from whole blood of the patient (i.e., the same patient from which the tissue sample has been resected), for example, using density centrifugation, using cell preparation tubes (CPTs), using SepMate tubes, or by any other methodology. If exposure to isolated PBMCs is desired at decision block 212, the method can proceed to process block 214 where the isolated PBMCs can be introduced to the tissue sample via addition to the flowing perfusate. In such an example, the isolated PBMCs may be tagged (e.g., using a nanoparticle tracking dye) prior to introduction to the tissue sample so as to aid in differentiation from immune cells that reside in the TME of the sample. The introduction of PBMCs may be part of a cancer treatment that increase a number or ability of circulating immune cells to target the tumor. The method can then return to process block 210 for perfusate circulation, such that the PBMCs are transported to the tissue sample in the perfusion chamber via the flowing perfusate for interaction with cells of the sample.

In some embodiments, after process block 214, the continuing to circulate perfusate of process block 210 can include continuously or periodically sampling the perfusate (e.g., for subsequent testing) or continuously or periodically detecting a substance within the flowing perfusate. For example, perfusate can be periodically sampled and tested to determine levels of one or more biomarkers therein. For example, in some embodiments, levels of transforming growth factor beta (TGF-β) in the flowing perfusate can be measured over time to assess response of the tissue sample, in particular a tumor therein, to an administered drug or treatment.

Otherwise, the method 200 proceeds to decision block 216, where it is determined if the tissue sample should be removed from the perfusion, for example, for imaging or treatment. For example, the tissue sample could be imaged after being exposed to the drug and/or PBMCs to assess the effect on various cells of the tissue. The determination of imaging at decision block 216 can be based on timing (e.g., waiting a period of time after initial drug exposure), a status of the tissue sample (e.g., whether the tissue sample is to serve as an experimental control), or any other criteria. If imaging is desired at decision block 216, the sample platform can be removed from the perfusion chamber and the method 200 can proceed to process block 218. If instead treatment is desired at decision block 216, the sample platform can be removed from the perfusion chamber and the method 200 can proceed to process block 224.

Alternatively or additionally, decision block 216 can involve determining if the tissue sample should be permanently removed, for example, for an analysis that involves tissue dissociation. For example, in some embodiments, the sample platform can be removed from the perfusion chamber, and the tissue sample removed from the sample platform. The tissue sample can then be analyzed, for example, by flow cytometry, multi-omics profiling methods, etc.

If temporary or permanent removal of the sample platform from the perfusate chamber is not desired at decision block 216, the method 200 can return to process block 210, where the circulation of perfusate continues.

When imaging is desired, after the sample platform is removed from the perfusion chamber, the tissue sample is prepared for imaging at process block 218. For example, the preparation can include one or more staining procedures (e.g., hematoxylin and eosin (H&E), immunohistochemical CD3, immunohistochemical CD20, immunohistochemical CD68, antigen Ki-67, etc.). During the preparation of process block 218, the tissue sample is not removed from the sample platform. Rather, in some embodiments, process block 218 may employ an exposure platform holder to perform the staining of the tissue sample, for example, using the platform holder of FIG. 1G. The method 200 can proceed to process block 220, where the sample platform with tissue sample is temporarily mounted on an imaging platform holder for imaging, for example, the imaging platform holder of FIG. 1D. As noted above, the imaging platform holder can be used to suspend the tissue sample within a fluid (e.g., plasma and/or culture medium) while being imaged and allows for convenient change in height of the tissue sample with respect to focal plane of the imaging system.

The method 200 can proceed to process block 222, where the tissue sample supported by the imaging platform holder is imaged. For example, the imaging system can comprise a microscope system, such as a confocal microscope, and the imaging platform holder can be constructed to rest on a horizontal sample stage of the microscope. The imaging system can be configured to perform any type of known interrogation technique or imaging modality, such as, but not limited to, confocal microscopy, fluorescence microscopy, two-photon or multiphoton microscopy, time-lapse microscopy (e.g., for live cell imaging), phase contrast microscopy, holotomography, etc.

Once imaging is completed, the method 200 can proceed to decision block 228, where it is determined if the tissue sample should be returned to the perfusion chamber for continued viability and/or testing. The determination at decision block 228 can be based on timing (e.g., a predetermined lifetime of the sample or experimentally determined maximum lifetime of the sample), a status of the tissue sample (e.g., whether imaging reveals the sample is no longer viable), or any other criteria. If return is desired at decision block 228, the sample platform can be removed from the imaging platform holder, and the method 200 can return to process block 208, where the sample holder is reinstalled in the perfusion chamber.

When treatment is desired, after the sample platform is removed from the perfusion chamber at decision block 216, the method 200 can proceed to process block 224, where the tissue sample can be temporarily mounted on an exposure platform holder, for example, the exposure platform holder of FIG. 1F or 1G. The method 200 can proceed to process block 226, where the tissue sample supported by the exposure platform holder is subjected to a treatment. As noted above, the exposure platform holder can be used to suspend part or all of the tissue sample within a fluid (e.g., drug) to provide such treatment. For example, in some embodiments, the fluid is a chemotherapeutic agent, and process block 226 comprises immersing part of the tissue sample within the chemotherapeutic agent for a predetermined period of time (e.g., 60-90 minutes). In some embodiments, the fluid can be heated (e.g., to a temperature at or slightly higher than normal body temperature of 37° C., for example, 42° C.) and/or agitated (e.g., stirring) during the immersion.

Once treatment is completed, the method 200 can proceed to decision block 228, where it is determined if the tissue sample should be returned to the perfusion chamber for continued viability and/or testing. The determination at decision block 228 can be based on timing (e.g., a predetermined lifetime of the sample or experimentally determined maximum lifetime of the sample), a status of the tissue sample, or any other criteria. If return is desired at decision block 228, the sample platform can be removed from the exposure platform holder, and the method 200 can return to process block 208, where the sample holder is reinstalled in the perfusion chamber.

Otherwise, the tissue sample can be discarded at 228 when it is determined that return is not desired. In some embodiments, the sample platform may be a disposable component, in which case the tissue sample and sample platform can be disposed of as a unit. In other embodiments, the sample platform may be a reusable component, in which case the tissue sample is dismounted from the tissue mount section and discarded. The sample platform can then be sterilized (e.g., via autoclave) for use with a subsequent tissue sample at 206.

Alternatively or additionally, after imaging in process block 222 or treatment in process block 226, it can be determined at decision block 228 to refrain from returning the tissue to the perfusion chamber in order to perform further analysis. For example, the tissue can be removed from the sample platform, subject to a chemical fixation process (e.g., using formaldehyde or glutaraldehyde), and then embedded within optimal cutting temperature (OCT) compound for subsequent frozen tissue sectioning (e.g., using a microtome-cryostat). The tissue sections can then be stained for complex imaging, such as immunohistochemistry (IHC) imaging (e.g., multiplex immunofluorescence imaging). Other analyses are also possible according to one or more contemplated embodiments.

Although FIG. 2 illustrates a particular order for blocks 202-228, embodiments of the disclosed subject matter are not limited thereto. Indeed, in certain embodiments, blocks 202-228 may occur in a different order than illustrated or simultaneously with other blocks. For example, the circulation of perfusate in process block 210 may be a substantially continuous process (e.g., interrupted only for periodic replacement of perfusate) and thus occurs at a same time as the process block 208, process block 214, and/or the removing of decision block 216. In another example, the imaging of process block 222 may occur prior to or after any addition of drugs to perfusate in process block 214, or prior to or after any ex chamber treatment in process block 226.

### Example 1—System

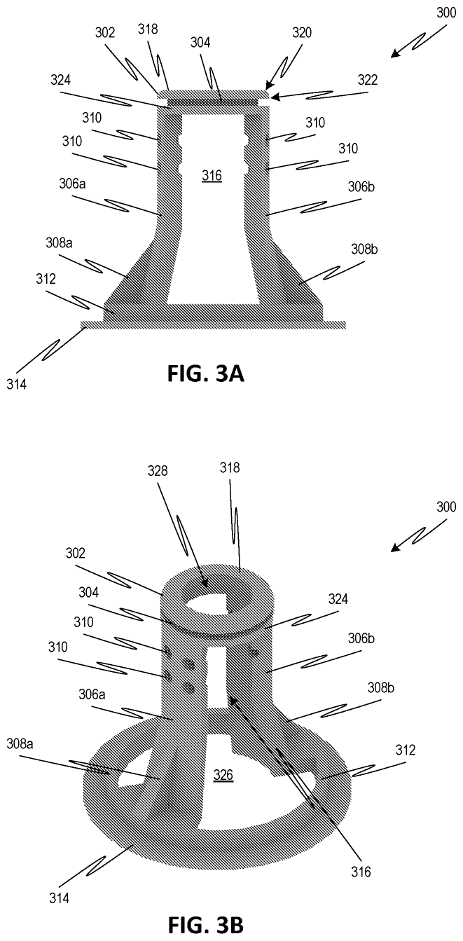

FIGS. 3A-3B illustrate an example construction for a sample platform 300. The sample platform 300 has a tissue mount section formed by a top annular member 302 and a chamber mount section formed by a flange 314 and cylindrical bottom member 312. Extending through the top annular member 302 is a through-hole 328, which exposes a backside of the tissue sample once provided on the tissue mount section. The top annular member 302 includes a circumferential groove 322 that separates an upper portion 318 from a lower portion 324. The circumferential groove 322 is designed to receive a suture, rubber band, or other mechanical attachment so as to retain the tissue sample to the tissue mount section. The periphery of the tissue sample would thus overhang the upper portion such that it extends over the groove 322 so that the mechanical attachment can clamp the periphery of the sample to the groove 322. To minimize or at least reduce damage to the tissue sample caused by sharp or pointed edges, at least the radially outer edge 320 of the upper portion 318 may be rounded or chamfered.

In some embodiments, the chamber mount section has an opening 326 that extends through flange 314 and cylindrical bottom member 312. In other embodiments, the chamber mount section may be substantially solid, for example, with flange 314 as a solid disk and/or bottom member 312 as a solid cylinder. The tissue mount section is connected to the chamber mount section by a pair of arms 306a, 306b, thereby spacing the tissue mount section from the chamber mount section along an axial direction of the sample platform 300. The arms 306a, 306b can be positioned on opposite sides of through-hole 328, thereby forming an open region 316 therebetween which communicates with the open through-hole 328 and opening 326.

Upper ends of arms 306a, 306b connect to lower portion 324 of the tissue mount section, while lower ends of arms 306a, 306b connect to the cylindrical bottom member 312. In some embodiments, each arm can have a respective support portion 308a, 308b at the lower ends and connected to the cylindrical bottom member 312. The support portions 308a, 308b may improve the rigidity and/or reliability of arms 306a, 306b. Each arm 306a, 306b can also include one or more through-holes 310 in a region proximal to the tissue mount section. Each through-hole 310 can extend through a thickness of the respective arm 306a, 306b to communicate with open region 316, thereby improving perfusate access to the backside of the tissue sample. In some embodiments, the through-holes 310 can be replaced by a single large hole (e.g., a rectangular window), a plurality of smaller holes (e.g., an array or mesh), or any other configuration of opening or openings.

In some embodiments, flange 314 can include one or more surface features (e.g., protrusion or recess) designed to cooperate with corresponding surface features of the holder (e.g., top circular disk 609 of lid 608) in order to lock, or at least releasably position, the sample platform in a predetermined orientation within the perfusion chamber. For example, in some embodiments, the predetermined orientation of the sample platform 300 is such that an upper portion of arms 306a, 306b (e.g., having through-holes 310) and/or open region 316 are substantially parallel to a direction of the perfusate flow through the perfusion chamber (e.g., in a top down plan view).

In a fabricated example, the flange 314 had an outer diameter of 18.6 mm, the upper portion 318 had an outer diameter of 9.56 mm, the inner diameter of the through-hole 310 was 6.68 mm, the groove 322 was indented from the upper portion outer diameter by 0.5 mm, the arms 306a, 306b were 16.75 mm in length along the axial direction and 4.78 mm in width, and each arm 306 had four through-holes 310 of 1 mm diameter. Other dimensions are also possible according to one or more contemplated embodiments. For example, a diameter and/or thickness of upper portion 318, a diameter of lower portion 324, and/or a width of groove 322 (e.g., in a direction extending between the upper portion 318 and the lower portion 324) can be increased from the above-noted exemplary dimensions to assist in attaching the tissue sample to the sample platform. For example, in some embodiments, the diameter of upper portion 318 can be made larger than that of the lower portion 324, such that the top annular member 302 is mushroom-shaped in side view.

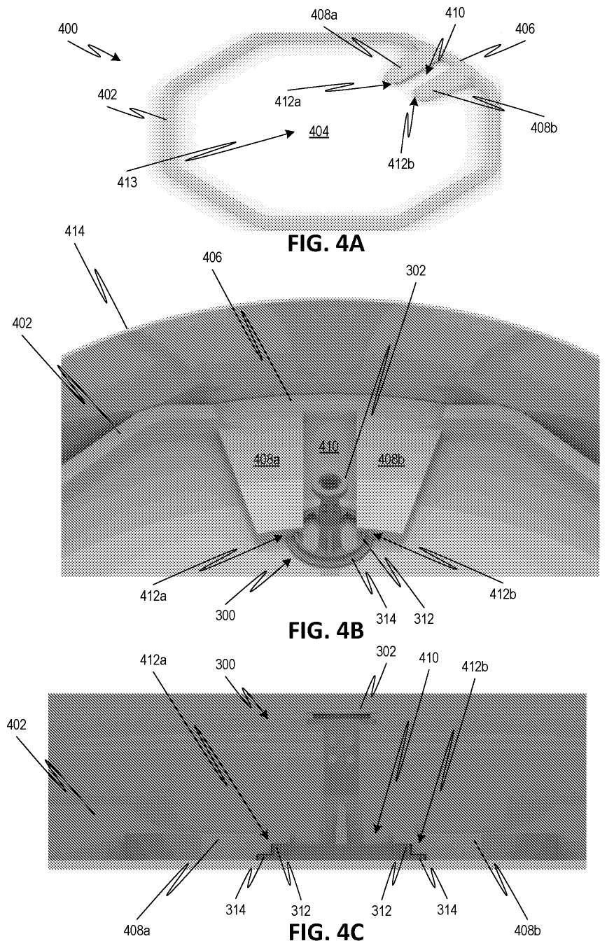

In some embodiments, a jig can be used to hold and stabilize the sample platform during the mounting of the tissue sample thereto. For example, FIGS. 4A-4C illustrate an exemplary mounting jig 400 (also referred to as a tying apparatus) that can be used with sample platform 300. The mounting jig 400 can have an octagonal-shaped wall 402 surrounding a central open region 404 and designed to fit within a circular dish (e.g., Petri dish). The flat portions of the wall 402 can abut the circumferential wall 414 of the circular dish so as to prevent rotation of the jig 400. At least one section 406 has a fixture for securing the sample platform 300. In particular, the fixture includes a pair of arms 408a, 408b extending from a periphery of the jig into open region 404. An underside of each arm 408a, 408b includes a grooved feature 412a, 412b that matches a shape of the chamber mount section, in particular, the flange 314 and cylindrical bottom member 312. The arms 408a, 408b are spaced from each other by a gap 410 designed to accommodate the arms and tissue mount section of the sample platform 300 therein. Of course, more than one section of the jig wall 402 can be provided with fixtures. For example, each wall section may be provided with a respective fixture (e.g., 8 total) for mounting tissue samples to multiple sample platforms using a single setup. Alternatively or additionally, the fixture can be moved from a periphery of the jig to a location within open region 404. For example, a pair of support arms can extend diametrically across jig 400 to retain arms 408a, 408b in a similar configuration as illustrated in FIG. 4A but at or proximal to a central region 413 of the jig 400. Other constructions and orientations for the fixture of jig 400 are also possible according to one or more contemplated embodiments.

In use, the jig 400 is first inserted into a circular dish, with portions of walls 402 in contact with a circumferential wall 414. The sample platform 300 is then placed within open region 404 at a radially innermost end of arms 408a, 408b. The chamber mount section of the sample platform 300 is aligned with the grooves 412a, 412b of the arms 408a, 408b, and the sample platform 300 is slid radially outward to engage with the grooves 412a, 412b, as shown in FIGS. 4B-4C. A tissue sample can then be draped over the top surface of the tissue mount section and secured thereto using one or more mechanical attachments. After the tissue sample has been mounted, the tissue assembly constituted by the tissue sample and sample holder can be slid radially inward until it disengages with grooves 412a, 412b, after which the tissue assembly can be removed from the setup for use (e.g., installation in the perfusion chamber).

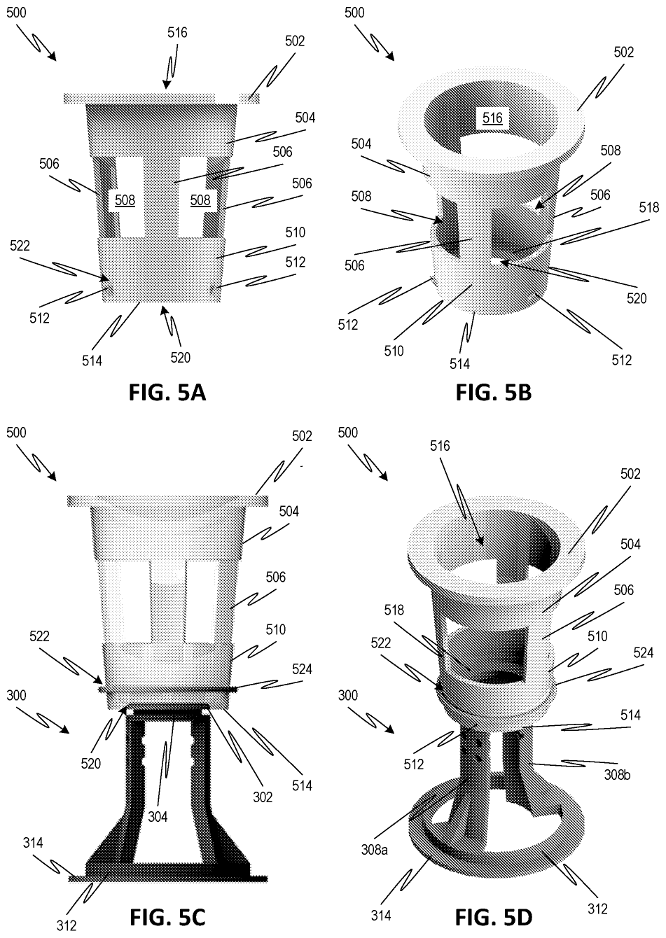

As noted above, in some embodiments, the mechanical attachment of the tissue sample to the tissue mount section may be by way of an annular flexible member that applies a radial compressive force to the periphery of the tissue sample, for example, a rubber band. FIGS. 5A-5D illustrate an exemplary applicator 500 that can optionally be used to assist with installing the rubber band to the tissue sample. The applicator 500 can have a truncated cone shape that tapers from top portion 504 to bottom portion 510. The top portion 504 can have a flange 502 at a top end thereof, the flange 502 surrounding a central opening 516. The top portion 504 can be connected to the bottom portion 510 via three arms 506, which are spaced from each other about the circumference of the applicator by intervening access windows 508 that communicate with central opening 516. Bottom portion 510 can include stops 512 disposed at intervals around its circumference proximal to the bottom end 514. The stops 512 thus define a region 522 where a rubber band 524 can sit in anticipation of deployment to the tissue sample on the sample platform 300. Bottom portion 510 has a central opening 520 surrounded by an internally extending flange 518, which is spaced from bottom end 514 by an amount corresponding to a location of the groove 304 of the sample platform.

In use, a tissue sample is draped over the top annular member 302 of the sample platform 300, and then the bottom end 514 of the applicator is positioned over and into contact with the tissue sample (e.g., as suggested by the configuration shown in FIGS. 5C-5D). In particular, the tissue sample and annular member 302 are inserted into the central opening 520 at the bottom end 514 until the tissue sample abuts the internally extending flange 518, whereby the bottom end 514 may be aligned with (or at least close to) groove 304. With the applicator 500 retained in this position, the rubber band 524 can then be moved from region 522 until it leaves end 514 for the sample platform 300. For example, the rubber band 524 can be advanced by rolling over the outer circumferential surface of the applicator 500, by sliding over the circumferential surface of the applicator 500, and/or by gently flicking until it settles over the tissue sample at groove 322. The use of applicator 500 can hasten preparation of sample platform 300 by making the process efficient while minimizing, or at least reducing, manipulation that may otherwise cause damage to the tissue. The open design of the applicator 500 (e.g., via central opening 516 and/or access windows 508) can allow for visualization of the tissue as it is secured over the sample platform 300.

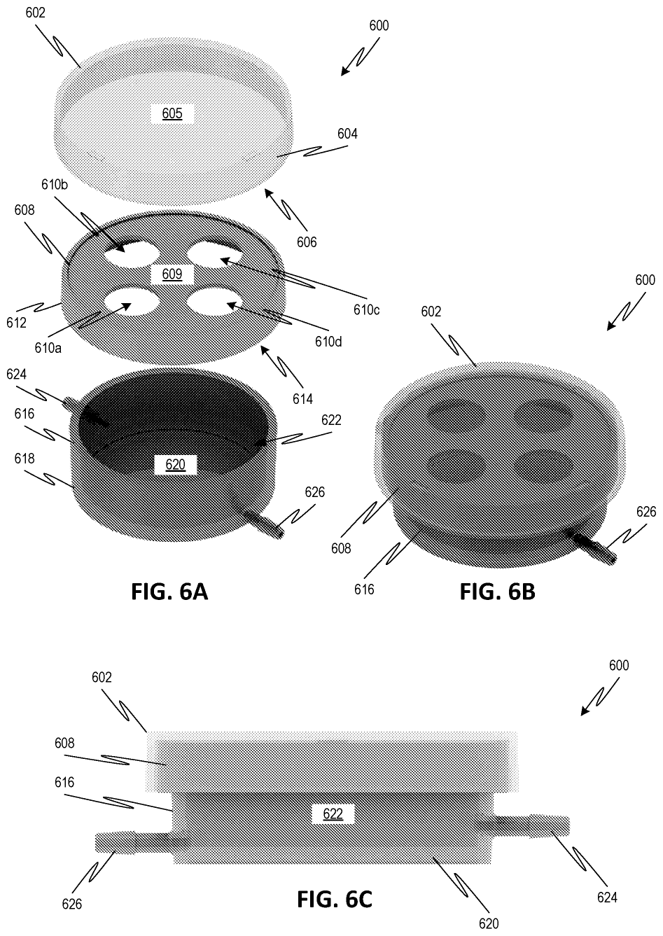

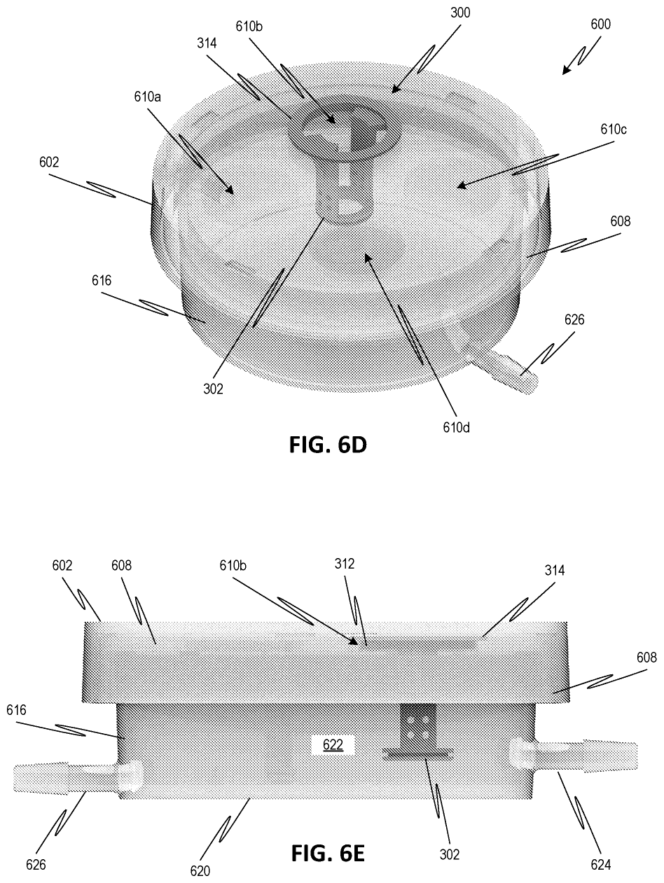

FIGS. 6A-6E illustrate an example construction for a perfusion chamber 600. The perfusion chamber 600 includes a bottom dish 616 having an internal volume 622 defined by a substantially cylindrical wall 618 extending from a circumference of a bottom circular disk 620. An inlet port 624 can extend through cylindrical wall 618 on one side of internal volume 622, while an outlet port 626 can extend through cylindrical wall on an opposite side of internal volume 622. As noted above, the inlet and outlet ports can be at different heights with respect to bottom circular disk 620. For example, the inlet port 624 may be farther from bottom disk 620 than the outlet port 626. The inlet port 624 may also be at a level closer to that of the tissue sample when the sample holder in installed in the perfusion chamber 600, while outlet port 626 may be at a level farther from that of the tissue sample, as shown in FIG. 6E. In some embodiments, when OGB structures (e.g., disks) are used, a bottom surface of the perfusion chamber 600 can have recesses (not shown) for receiving the OGB structures therein. For example, the recesses can correspond in location (e.g., be aligned with) and/or size to the recesses 610a-610d in lid 608, as described below.

The perfusion chamber 600 can also include a lid 608 (also referred to as a platform holder) having an internal volume 614 defined by a substantially cylindrical wall 612 extending from a circumference of a top circular disk 609. The cylindrical wall 612 has an inner diameter greater than an outer diameter of cylindrical wall 618, such that lid 608 can fit over a top end of the dish 616 to enclose internal volume 622. The top circular disk 609 of the lid 608 includes four recesses 610a-610d extending therethrough and communicating with internal volume 614. Recesses 610a-610d are designed to receive respective sample platforms therein, in particular, allowing the tissue mount section to pass therethrough. However, a diameter of each recess 610 is smaller than a diameter of flange 314 of the chamber mount section, such that the sample platform 300 is releasably coupled to the lid 608 while suspending the rest of the sample platform 300, including the mounted tissue sample, within the internal volume 622, as shown in FIGS. 6D-6E.

In some embodiments, the lid 608 and the dish 616 can comprise one or more cooperating features that align and/or retain (e.g., lock) the lid 608 (and the sample holders supported thereby) in a predetermined orientation with respect to the dish 616 and/or contents thereof. For example, similar to the configuration illustrated in FIG. 1C, the lid 608 can have one or more protrusions that fit within respective recesses of the dish 616, and/or the dish 616 can have one or more protrusions that fit within respective recesses of the lid 608. The cooperating features of recesses and protrusions act to position the lid 608 and the supported sampled holders in a fixed orientation with respect to the dish 616, for example, with respect to a direction of perfusate flow in the perfusion chamber 600. Moreover, in some embodiments, the cooperating features can resist rotation of the lid 608, for example, due to forces of the perfusate flow on the sample platform supported by the lid 608. In some embodiments, the cooperating features can be keyed, such that only one orientation is possible for the lid installed on the dish. For example, in some embodiments, the lid 608 can have a single protrusion, and the dish 616 can have a single recess. In another example, the lid 608 can instead have a pair of protrusions that are not diametrically aligned (e.g., with respect to the top circular disk 609 of the lid 608 or bottom circular disk 620 of dish 616), and the dish 616 can have a corresponding arrangement of recesses. Other configurations for such cooperating features to align and/or retain the lid are also possible according to one or more contemplated embodiments.

In some embodiments, the perfusion chamber 600 can also include a cover 602 having an internal volume 606 defined by a substantially cylindrical wall 604 extending from a circumference of a top circular disk 605. The cover 602 can be designed to fit over the lid 608, thereby protecting open ends of the chamber support sections of sample platforms 300 inserted into recesses 610a-610d of the lid 608. In some embodiments, the cover 602, lid 608, or both can include one or more spacers that hold the top circular disk 605 spaced away from the flange 314 of each sample platform 300 retained in recesses 610a-610d of lid 608, for example, to prevent adhesion of the cover 602 to the sample platforms, the lid, or both. Alternatively or additionally, the spacer can be a separate member (e.g., an O-ring) disposed between cover 602 and lid 608.

In a fabricated example, the lid 608 had an outer diameter of 61.49 mm and a length of 9 mm, the wall 612 of the lid flared radially outward by about 2ⁿ (e.g., forming an angle of 92° with top disk 609), a thickness of top disk 609 was 2 mm, each recess 610a-610d had a diameter of 15.9 mm, the dish 616 had an outer diameter of 56.71 mm and a length of 19.35 mm, the wall 618 of the dish flared radially outward by about 2° (e.g., forming an angle of 92° with bottom disk 620), a bottom of outlet 626 was 2 mm above the bottom disk 620, and a bottom of inlet 624 was 5 mm above the bottom disk 620. Other dimensions are also possible according to one or more contemplated embodiments.

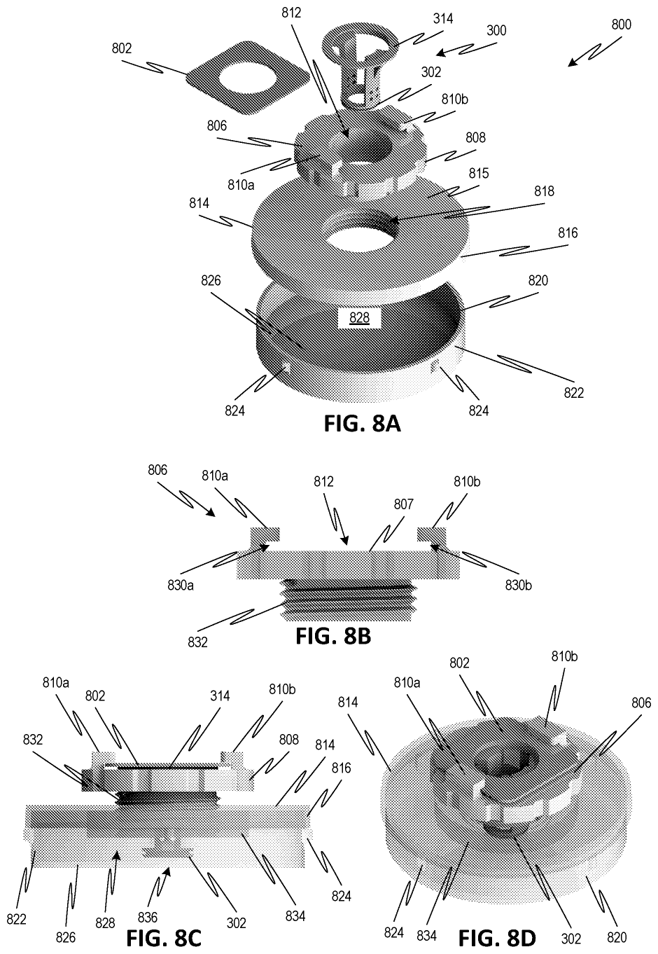

FIGS. 8A-8D illustrate an example construction for an imaging platform holder 800 for imaging of the tissue sample mounted on the sample platform 300. The imaging platform holder 800 has a locking member 802, a first member 806, a second member 814, and a dish 820. The dish 820 has an internal volume 828 defined by a substantially cylindrical wall 822 extending from a circumference of a bottom circular disk 826. The bottom circular disk 826 may be formed of a transparent material or may include at a center thereof (e.g., at 836 in FIG. 8C) a viewing port formed of a transparent material. The dish 820 is configured to hold a fluid, such as plasma and/or culture medium, therein to nourish the tissue sample during imaging. A plurality of stops 824 can be disposed on an exterior surface of the cylindrical wall 822.

The second member 814 has a substantially cylindrical wall 816 extending from a circumference of a top circular disk 815. A substantially cylindrical shaft 834 extends from a bottom surface of the top circular disk 815. A central threaded opening 818 extends through both the top circular disk 815 and the cylindrical shaft 834. The cylindrical wall 816 has an inner diameter greater than an outer diameter of cylindrical wall 822, such that second member 814 can fit over a top end of the dish 820 to enclose internal volume 828, with a bottom end of cylindrical wall 816 resting on stops 824 of the dish 820.

The first member 806 similarly has a top circular disk 807 and a substantially cylindrical shaft 832 extending from a bottom surface of the top circular disk 807. A central opening 812 extends through both the top circular disk 807 and the cylindrical shaft 832. The cylindrical shaft 832 has a threaded circumferential surface that corresponds to threads of central opening 818 of the second member 814. The shaft 832 of the first member 806 is thus coupled with opening 818 of the second member 814 and rotated therein to move the first member 806 axially with respect the second member 814 and the dish 820. The first member 806 may optionally include a knurled circumferential edge 808 to facilitate rotation by a user. By rotating the first member 806 with respect to the second member 814, the tissue sample can be moved axially to or from the bottom disk 826 of dish 820, for example, to move a different portion of the tissue sample into focus for imaging.

Central opening 812 is designed to receive a sample platform 300 therein, in particular, allowing the tissue mount section (e.g., top annular member 302) to pass therethrough. However, a diameter of opening 812 is smaller than a diameter of flange 314 of the chamber mount section, such that the sample platform 300 is releasably coupled to the first member 806 while suspending the rest of the sample platform 300, including the mounted tissue sample, within the internal volume 828, as shown in FIGS. 8C-8D. The top disk 807 of first member 806 also includes a pair of L-shaped securing arms 810a, 810b on opposite sides of central opening 812. The securing arms 810a, 810b form respective gaps 830a, 830b with a top surface of top disk 807, as shown in FIG. 8B.

In some embodiments, the height of the gap 830a, 830b is greater than a thickness of the flange 314. Alternatively or additionally, a distance between facing ends of the securing arms 810a, 810b may be greater than an outer diameter of flange 314. Accordingly, the securing arms 810a, 810b alone may not adequately retain the sample platform 300 to the imaging platform holder 800, for example, where fluid within dish 820 generates an axial force due to buoyancy of the sample as the first member advances toward the dish 820. Locking member 802 can thus be provided between the securing arms 810a, 810b and the exposed surface of the flange 314 and form a transition fit (e.g., tight fit, similar fit, or fixed fit) or an interference fit (e.g., press fit) that secures the flange 314, and thereby the sample platform 300, to the first member 806, as shown in FIGS. 8C-8D.

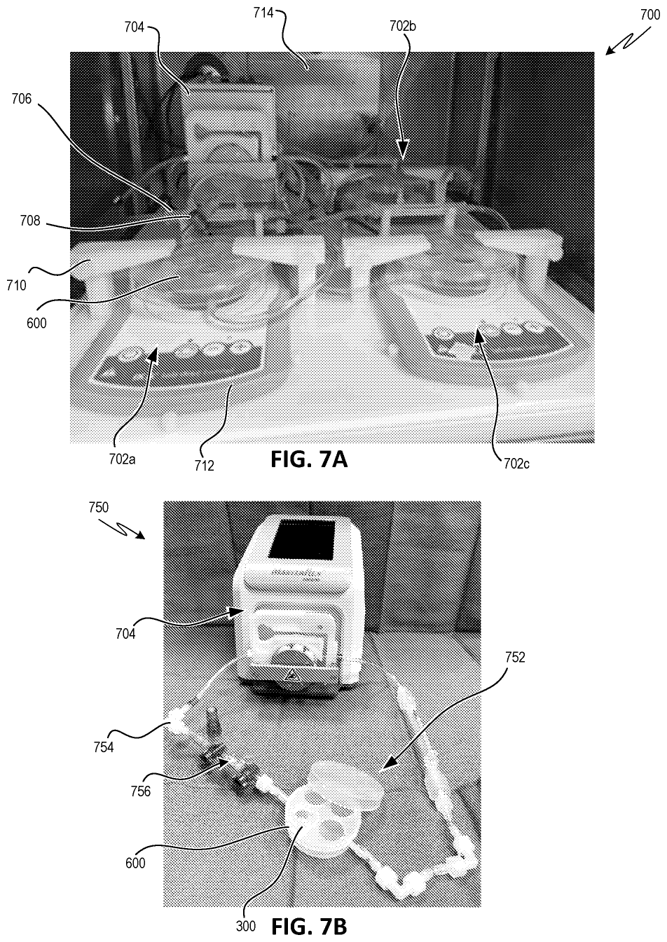

FIG. 7A shows an example of a fabricated system 700 according to embodiments of the disclosed subject matter, in particular, including sample platform 300 and perfusion chamber 600. The system 700 included three separate subsets 702a-702c that shared a common peristaltic pump 704 (ISM828 Reglo analog variable speed pump, sold by Ismatec, a division of Cole-Parmer GmbH, Wertheim, Germany) to move perfusate through respective fluid circuits and perfusion chambers 600. In alternative configurations, only two separate subsets share a common peristaltic pump. The fluid circuits can be formed of laboratory grade tubing (Tygon LMT-55 tubing, sold by Ismatec, a division of Cole-Parmer GmbH, Wertheim, Germany). Each fluid circuit had a respective oxygenator 706 (silicone hollow fiber membrane with 1000 cm² surface area, sold by PermSelect of Ann Arbor, Michigan) connected thereto, with an outlet of the oxygenator 706 connected to a respective CO₂ regulatory valve 708. The fluid circuits further included in-line 3-way valves for continuous infusions and perfusate sampling. Each perfusion chamber 600 was disposed over a magnetic stirrer plate 712 (Lab Disc, sold by IKA Works, Inc., Wilmington, NC) for actuating a magnetic stirrer bar contained within the perfusion chamber 600. In alternative configurations, a magnetic stirrer plate 712 that is resistant to humidity or other environmental conditions within incubator 714 can be used, such as the Cimarec-i microstirrer (ThermoFisher Scientific). A pair of arms 710 held a housing containing the perfusion chamber 600 in place over the respective magnetic stirrer plate 712. The entire setup was contained within an incubator 714, which maintained a temperature of 37° C. In alternative configurations, the magnetic stirrer plate 712 and associated magnetic stirrer bar can be omitted altogether.

FIG. 7B shows another example of a fabricated setup 750 according to embodiments of the disclosed subject matter, in particular, including sample platform 300 and perfusion chamber 600. In the illustrated example, system 750 has a single subset 752 connected to a peristaltic pump 704, although additional subsets 752 can be provided to share the same peristaltic pump. Pump 704 recirculates perfusate through fluid circuit 754 and perfusion chambers 600. In the illustrated example, fluid circuit 754 can have elbow connectors and reduced tubing lengths to minimize a total perfusate volume flowing through the system, for example, about 12-15 mL. The fluid circuit 754 further includes in-line 3-way valves 756 for infusions and/or perfusate sampling. Similar to the system of FIG. 7A, the entire setup of FIG. 7B can disposed within an incubator to maintain a temperature within perfusion chamber 600 of about 37° C. Unlike the configuration of FIG. 7A, the setup of FIG. 7B lacks any active mixing features (e.g., magnetic stirrer bar). Instead, setup 750 relies solely on passive features to provide mixing, in particular, the vertically-offset arrangement of inlet and outlet ports of the perfusion chamber 600. Utilizing such passive mixing features instead of active mixing features may better avoid damage to the tissue sample retained on the sample platform 300.

Multiple solid tumors commonly manifest as metastatic disease to the mesothelial surface (peritoneum, pleura, liver capsule). Mesothelium is thin, translucent and is commonly resected both with open and laparoscopic/thoracoscopic surgery as part of both staging and standard treatment algorithms. The thin tumors of mesothelial surfaces enable oxygen diffusion and facilitates real-time or recent (e.g., within a few minutes) imaging interrogations. Accordingly, tumor-bearing mesothelium was suspended in the perfusate using the above-described platforms, which were designed to maximize gas exchange and facilitate imaging.

The perfusate was composed of patient blood-type matched human fresh frozen plasma (FFP), Dulbecco's Modified Eagle Media (DMEM) with GlutaMAX (ThermoFisher Scientific), glutathione (1 mM), insulin (Novolin R 100 units/mL), 5% dextrose (0.25 mL per 30 mL) and penicillin-streptomycin (Pen-Strep). The perfusate was prepared in a sterile fashion inside a biosafety cabinet. A one-to-one ratio of DMEM to FFP was utilized, with addition of 0.5 mL of insulin (Novolin R 100 units/mL) and Pen-Strep for a total volume of 30 mL. Alternatively, this volume permits plasma to be donated by the individual patient from whom tumor is used (as opposed to the blood bank), which can facilitate biomarker and correlative science discovery, and is acceptable from a drug utilization perspective. In some configurations, the volume can be reduced further by decreasing sizes of the perfusion chamber, the sample platform, and/or associated fluid circuit (e.g., diameter, length, and/or shape of tubing between components of the system), such that a minimum total volume of perfusate for the system is 12-15 mL. In such configurations, the perfusate may be 100% autologous plasma (e.g., without culture media, although potentially supplemented with one or more drugs, hormones, and/or nutrients).

Humidified oxygen was used with the system in order to minimize evaporate losses (and the accompanying electrolyte derangements), and pH was controlled with the CO₂ pressure control valve on the venting outlet of the oxygenator to let off excess carbon dioxide. In alternative configurations, the perfusion chamber can also be provided with OGB structures to generate oxygen within the perfusate. In further alternative configurations, a humidified low-flow gas blender, such as MCQ Gas Blender 100 (sold by MCQ industries in Rome, Italy) can be used to supply a dynamic, customized mixture of oxygen and CO₂ gases.

To compensate for evaporative loss of water, sterile water and insulin were continuously infused at a rate of 0.25-0.75 mL/hour using a metered syringe injector. Although continuous infusion of insulin was used, any drug/hormone/growth factor/cells/etc. can be added to the system in a similar manner. To ensure maintenance of physiological parameters, point of care testing was conducted/carried out at interval timepoints with an iStat analyzer (sold by Abbott of Chicago, Illinois) to assess pH, oxygen, and metabolite levels, and appropriate adjustments were made, as needed.

Tissues introduced to the system were kept viable for up to four days. Every 24 hours, a complete perfusate exchange was performed. Experimental drugs were introduced into the perfusate as indicated. Tissue samples taken immediately upon extirpation from the patients were preserved in 10% neutral buffered formalin and served as a control (Day 0) for a given experiment. Throughout the four-day experiment, sample platforms were removed from the perfusion chamber at various endpoints to demonstrate viability and drug effects.

### Example 2

Sample platforms containing tumor-bearing and adjacent normal mesothelium tissues were assembled. The maintenance and preservation of the normal tissue can be important to prevent any uncertain effects on the adjacent tumor. Moreover, the potential side of effects of new agents could be estimated using normal mesothelial platforms run in parallel to tumor-bearing mesothelium. Peritoneum, liver capsule and pleura tissues were assembled and perfused using the system of FIG. 7A.

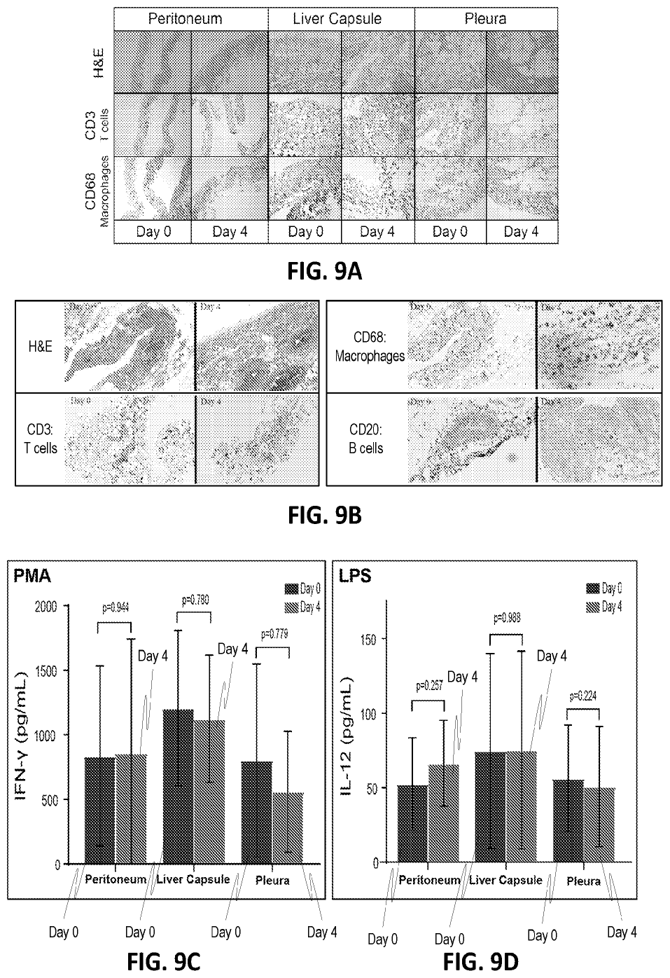

Using standard histologic evaluation (H&E), cellular preservation (95%) of samples at 96 hours was demonstrated as shown in FIGS. 9A-9B. In particular, the H&E stain displays preservation of normal collagen architecture and expected cell nuclear conservation. The normal mesothelial samples were then evaluated for immune cell populations (T cells, B cells, and macrophages) with standard immunohistochemistry (IHC) stains (CD3, CD20, and CD68). These IHC stains of the normal samples indicated preservation of cell numbers and morphology, as shown in FIGS. 9A-9B. In particular, the IHC stains demonstrate expected populations of immune cells through perfusion with the system.

With the goal of establishing a system that can evaluate immunomodulatory agents, it was sought to demonstrate that native immune cells can respond to stimulants in the system of FIG. 7A. Accordingly, phorbol 12-myristate 13-acetate (PMA) and lipopolysaccharide (LPS) were added to the perfusate in order to evaluate T cells and macrophages, respectively. In particular, the tissues on Day 0 and Day 4 were stimulated with PMA (10 ng/ml) or LPS (100 ng/ml) for twelve hours, after which the media was tested. Using IL-12 and interferon-γ (IFN-γ) as readouts, preservation of stimulatory capacity was demonstrated, as shown in FIGS. 9C and 9D.

### Example 3

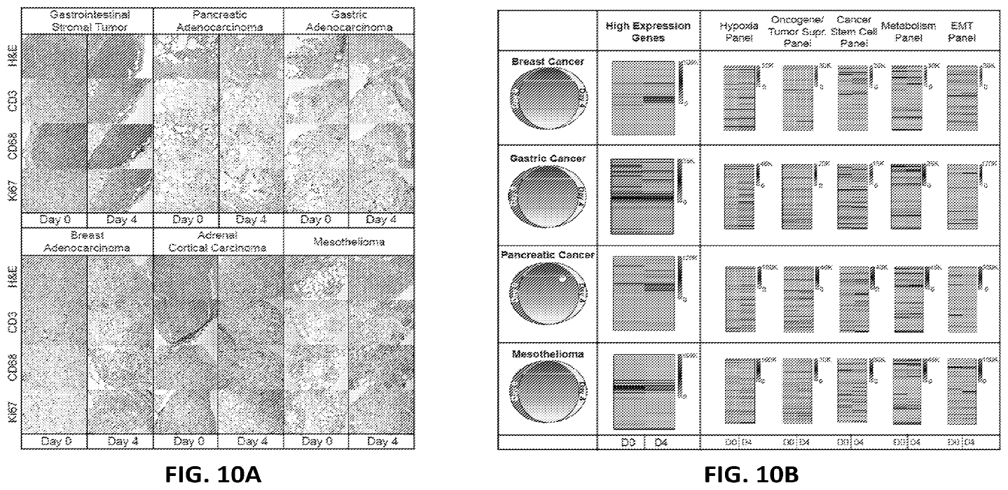

Using the setup of FIG. 7A, sample platforms were perfused for 96-hours from patients with variable cancer histologies. Using standard immunohistochemistry (IHC) techniques (i.e., H&E, CD3, CD68, and Ki67 stains), cellular and architectural preservation was demonstrated, as well as expected mitotic activity, as illustrated in FIG. 10A. Using PMA and LPS, preservation of stimulatory capacity of T cells and macrophages was also demonstrated. Next, it was shown that perfusion in the system of FIG. 7A does not substantially alter tumor transcriptomic profiles with the use of RNA sequencing, as shown in FIG. 10B. Genes associated with hypoxia, oncogenes/tumor suppressor, cancer stem cells, metabolism, and epithelial-mesenchymal transition were isolated from the sequencing data and demonstrated conservation of transcriptomics. These results ultimately demonstrate that tumor nodules in their native, functional microenvironment can be preserved ex vivo for 96 hours with respect to transcriptomic fidelity.

### Example 4

Any meaningful translational platform should be readily amenable to interrogation to understand why some cells within a tumor respond while others are ostensibly resistant. The design of the system of FIG. 7A allows tissue samples to be subjected to complex 3D imaging, including real-time, repeat time-lapse live imaging.

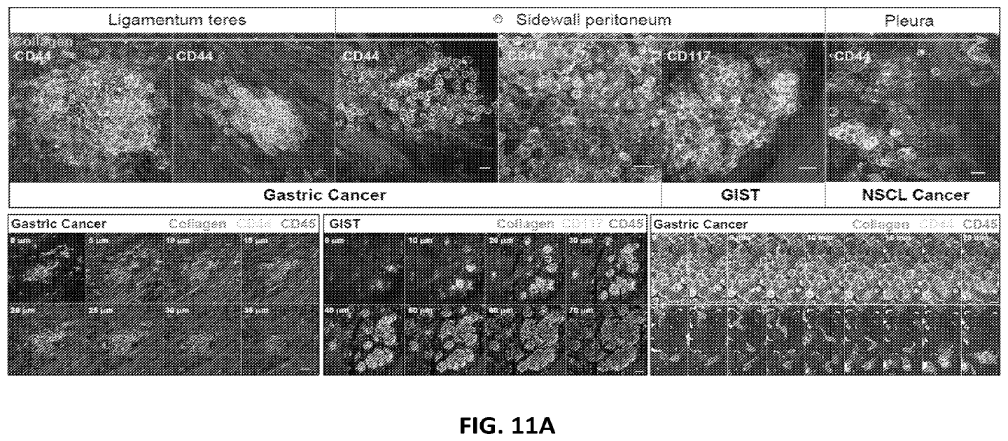

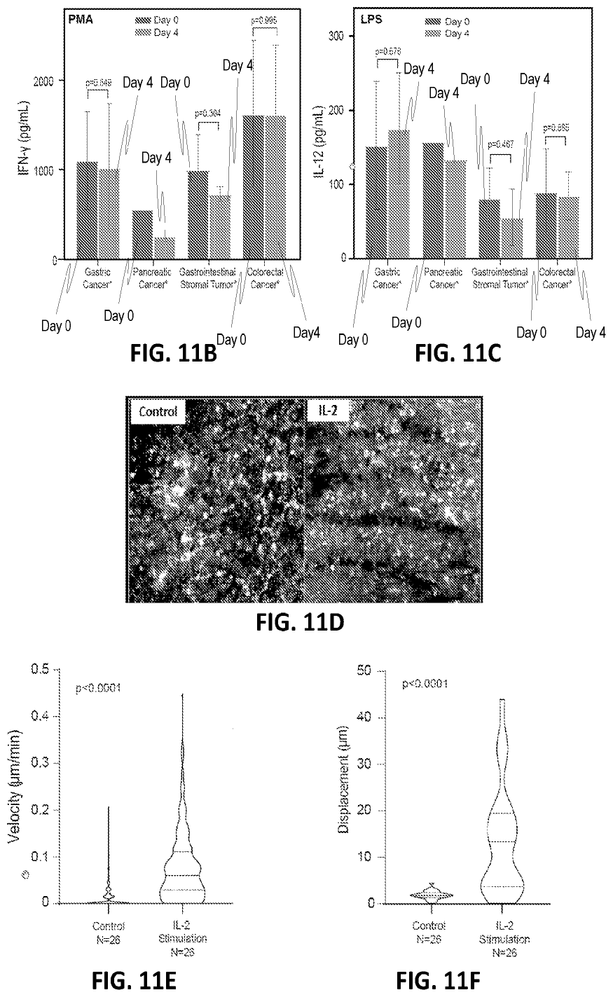

For example, FIG. 11A shows imaging results obtained for tissue samples cultured in system 700. Using commercially available fluorescently conjugated antibodies, nests of tumor cells (CD44) from various histologies embedded in collagen matrix with variable compactness were demonstrated, as shown in FIG. 11A. Maximum intensity projections of 2-photon live images were taken of cancerous biopsies of ligamentum teres, peritoneum and pleura from patients with gastric cancer, gastrointestinal stromal tumor (GIST) or non-small cell lung (NSCL) cancer secured on respective sample platforms following incubation with fluorescently tagged CD44 or CD117 antibodies (green) together with second harmonic generation (collagen, blue), where the bar represents 20 μm. The metastases cultured using system 700 retain their immune cell population.

FIG. 11A also shows Z-stack montages prepared from live images following incubation with Alexa488-conjugated CD44 or CD117 antibodies as well as Alexa594-conjugated CD45 antibody (bar represents 50 μm). As shown in the figure, immune cell population within metastases cultured in system 700 retain their mobility. FIG. 11A further shows a time series montage from live images taken in 2.5-min intervals of a gastric cancer biopsy following incubation with Alexa488-conjugated CD44 antibody and Alexa594-conjugated CD45 antibody (merged channels on top, CD45 in bottom panel).

With the application of a leucocyte marker (CD45), the relationship between tumor and immune cells becomes apparent. In particular, the GIST demonstrates “cold” tumor morphology with immune cells essentially excluded or surrounding nests of tumor cell. In contrast, the gastric tumor in FIG. 11A demonstrates interspersed immune cells within the tumor, possibly representing vulnerability to immune checkpoint inhibition.

Finally, immune cells can be tracked and monitored in the system of FIG. 7A. With the application of IL-2, a leucocyte activating cytokine, cell velocity and displacement is quantifiable. This can be an important metric for agents that aim to activate various immune subpopulations. For example, FIGS. 11B-11C show Interferon-γ and IL-12 secretion retained after 4 days in culture. Sample platforms in culture on Day 0 and Day 4 were stimulated with PMA (10 ng/ml) or LPS (100 ng/mL) for twelve hours and media was tested for Interferon-γ and IL-12 respectively across various histologies. FIG. 11D shows an increase in velocity and displacement of CD45-positive cells after addition of IL2 to the culture medium. FIGS. 11E-11F show that CD45 positive cells within the sample platform of FIG. 7A retain their ability to respond to IL2. In FIGS. 11E-11F, sample platforms bearing gastric cancer metastases to the peritoneum was evaluated via life imaging after 4 days in culture in system 700 of FIG. 7A.

Given a functional immune system, the use of full-length antibodies for cell recognition may result in unintended consequences. Accordingly, in some embodiments, fluorescently-labelled antigen-binding fragments (Fab fragments) and nanobodies can be used for various immune subpopulations instead of or in addition to full-length antibodies.

### Example 5

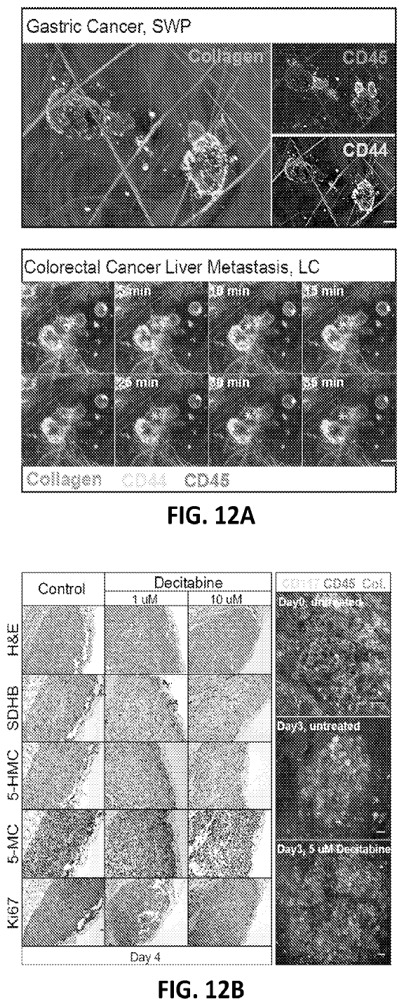

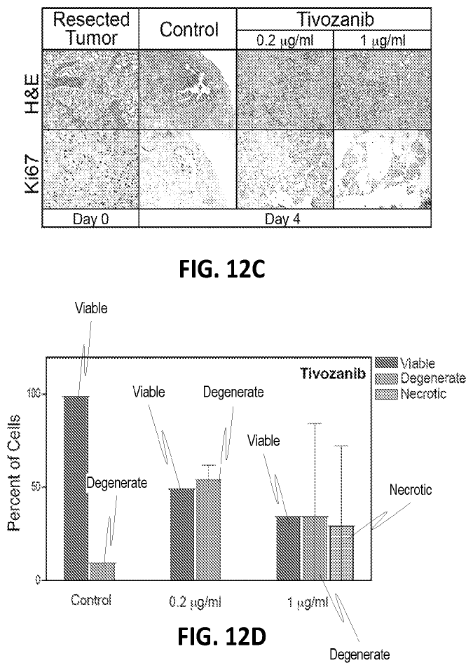

The system of FIG. 7A is well-suited to evaluate immunotherapeutic agents. Given that check-point inhibitors have made their way into the clinic for seemingly most solid tumors, such inhibitors were evaluated in the system of FIG. 7A using a tumor from a patient with gastric adenocarcinoma and a tumor from a patient with colorectal adenocarcinoma. Neither of these histologies have significant response rates to check-point inhibitors outside of select genomic subpopulations, which was not applicable to either patient in this example. FIGS. 12A-12D illustrates results of this example.

In FIG. 12A, it was shown that pembrolizumab stimulates binding of immune cells to cancer cells. In the top panel, sample platforms with sidewall peritoneum from a gastric cancer patient were incubated with immune checkpoint inhibitor pembrolizumab for 2 days followed by Alexa488CD44/Alexa594CD45 antibodies. Maximum projections of 2-photon images illustrate the intimate contact between CD45-positive cells and the cancer cells. The bottom panel provides a timeseries montage of images taken from liver capsule tissue mounted on sample platforms from a colorectal cancer patient with liver metastases incubated with 5 μg/ml of pembrolizumab for 4 hr. CD45-positive cells bound to CD44-positive cancer cell are shown undergoing apoptosis. Immune cell engagement was observed with tumors cells for both gastric and colorectal adenocarcinoma, and captured tumor cell lysis with the latter. It was also observed that some tumor cells appear unengaged by immune cells, which suggests that even among the ostensible non-responders there may be responses on a cellular level.

The system of FIG. 7A is also well-suited as a translational tool and/or as a tool for enrollment into clinical trials, especially where pre-clinical models are otherwise non-existent. For example, SDH-B deficient GIST is a rare subtype driven by epigenetic silencing of the gene, unresponsive to conventional agents (imatinib) and with no available cell lines or mouse models. Tissue from a patient with SDH-B deficient GIST was utilized in the system of FIG. 7A to investigate decitabine, a potential treatment option being evaluated for this population. Using early demethylation markers as a read-out, changes associated with decitabine treatment at physiologic and supraphysiologic doses were not detected. For example, FIG. 12B shows that decitabine has no effect on methylation status or proliferation activity of GIST. Sample platforms of tissue from the patient with GIST metastases to the peritoneum were cultured in perfusate supplemented with 0-10 μM decitabine. H&E staining of tissues harvested after 4 days in culture as well as IHC images using SDHB, 5-HMC, 5-MC and Ki67 antibodies suggested no changes in the methylation status or the proliferation activity. Stack projections of live images of control sample at day 0 and day 3 as well as a sample treated with 5 μM decitabine with fluorophore-conjugated antibodies against CD45 and CD117 also show no changes in the decitabine-treated tissue.

In another example, the system of FIG. 7A was used to identify a new target in patients with cholangiocarcinoma. The target is a kinase, SLK, that activates AKT signaling leading to aggressive tumor biology and poor patient outcomes. Although no SLK-specific inhibitor exists, the kinome profiles of available kinase inhibitors was evaluated, and tivozanib was identified. Using tissue from a patient with cholangiocarcinoma in the system of FIG. 7A, tivozanib was evaluated. The use of tivozanib demonstrated tumor cell degeneration at physiologic doses and frank necrosis at supraphysiologic concentrations. For example, FIGS. 12C-12D show that tivozanib treatment induces cell death in liver metastases. Resected cholangiocarcinoma tumor as well as liver capsule mounted on sample platforms treated with 0-1 mg/ml tivozanib were prepared for IHC, stained with H&E and Ki67 antibody. Cells in control and drug-treated platforms were evaluated for viability, degeneration and necrosis.

### Exemplary Methods

### Tissue Preparation

In any of the above discussed examples, tissue tested in system 700 was similarly prepared. For example, a specific tissue was procured directly from the operating room at the beginning of each operation and transported to the ex-vivo laboratory in pre-warmed Dulbecco's Modified Eagle Media (DMEM). Upon arrival to a sterile biosafety hood, the tissue was transferred into a larger cell-culture dish containing DMEM equilibrated to 37° ° C. using a heating plate, in order to prevent temperature fluctuations and warm ischemia to the tissue. Following visual inspection, excess adipose tissue was stripped carefully and mounted loosely over the small ring (e.g., top annular member 302) of the sample platform, with the mesothelial surface facing outward. Macroscopically visible tumor was positioned over the center of the platform before securing it in place with a 1-0 or 0 silk suture. The duration of the tissue preparation and mounting to the sample platform was limited to an average of five minutes. The sample platform was immediately transferred into the system 700, with the tissue side facing down within a sterile incubator (Thermo Heracell VIOS 160i CO₂ incubator) at 37° C. and 5% CO₂ for the duration of experimentation.

### Histopathologic Evaluation

In any of the above discussed examples, tissue from system 700 was subject to similar histopathologic evaluations. For example, the tissues were preserved in the 10% neutral buffered formalin, then through the tissue processor and embedded in paraffin and cut at 5-micron sections for hematoxylin and eosin (H&E) slides. Slides were prepared for H&E staining and immunohistochemistry, respectively to detect CD3 (T cells), CD68 (macrophages), Ki-67 and MIBI (mitotic activity). CD3 and CD68 were predilute antibodies as purchased, while Ki-67 and (MIB-1) were run at 1:200 dilution. All stains were done on an automated immunostainer (Ventana Benchmark Ultra). Viability on H&E was defined as preservation of normal tissue architecture, intact nuclei, and normal population of immune cells (qualitative and quantitative). Each sample was compared against day 0 control samples. Ki67 or stains were performed to assess proliferative activity of the tumor cells. Samples stained with Ki67 or MIB1 on day 4 were compared to day 0 samples to confirm no change in proliferative activity.

### Immune Cell Activation Testing

In any of the above discussed examples, tissue from system 700 was subject to similar immune cell activation testing. For example, in assessment of interferon-γ production, sample platforms were removed from the system 700 at 24 hour intervals for 4 days and placed in 2 mL of perfusate media and stimulated with recombinant human Interleukin-2 (IL-2) (1:1000 dilution), 50 ng/ml of phorbol 12-myristate 13-acetate (PMA), and 1 μg/mL of ionomycin. The tissue samples were allowed to incubate overnight. The following day the supernatant of each sample was harvested and stored at −80° ° C. Following collection of all samples, the supernatants were thawed and used for interferon-γ enzyme-linked immunosorbent assay (ELISA).

In preparation, flat-bottom 96-well plates were pretreated with 1 μg/ml of a human IFN-γ capture antibody, at 4° C. Immediately preceding the assay, the plates were blocked with 150 μl of 5% BSA in PBS for 30 min at room temperature. After removing the blocking solution, 50 μl of both the sample supernatants and the interferon-γ standard dilutions were added. Afterwards, 50 μl of 0.5 μg/ml biotinylated anti-human-IFN-γ detection antibody (50 μl/well, diluted in 5% bovine serum albumin (BSA) in phosphate buffer solution (PBS)) were added to each well. The plate was incubated for 60 minutes at room temperature, followed by four washes with ELISA buffer (PBS containing 0.05% Tween 20). 125 ng/ml streptavidin-horseradish peroxidase (150 μl/well, diluted in PBS with 5% BSA) was added and the plate was incubated again for 45 min at room temperature. The plate was then washed eight times with ELISA buffer and developed with TMB substrate (100 μl/well) in the dark. The reaction was stopped with 100 μl/well of 0.1 M H₂SO₄. Each ELISA plate was measured using a SpectraMax 190 microplate reader (Molecular Devices) and the associated software SoftMax Pro 6.2.2 (Molecular Devices).

In assessment of IL-2 production, sample platforms were removed from the system 700 at 24-hour increments for 4 days and placed in 2 mL of media and stimulated with Macrophage Colony Stimulating Factor (MCSF, 50 ng/mL) and lipopolysaccharide (LPS, 100 ng/ml). Macrophage function was assessed by cytokine bead capture of soluble IL-12p70 using the Legendplex Human Macrophage/Microglia kit. (BioLegend, San Diego, CA). The media was harvested and centrifuged at 6000×g for 60 seconds to separate debris from the supernatant. 25 μl of each respective sample supernatant was then added to a 96 well V-bottom plate followed by 25 μl of the assay buffer and 25 μl of the capture beads. The plate was then incubated for 2 hours at room temperature on a shaker to allow cytokine-bead binding. The plate was then centrifuged at 250×g for 5 minutes, inverted and flicked to remove the supernatant leaving the beads. The beads were then washed, followed by a second centrifugation at 250×g for 5 minutes and flicking of the wash buffer. 25 μl of the biotinylated detection antibody was then added to each well and incubated at room temperature, in the dark on a shaker, at 800 rpm. 25 μl of strepavidinphycoerythrin (SAPE) was added and the plate was placed on a shaker for 30 minutes. The plate was washed and flicked, and the beads were suspended in 150 μl of wash buffer. Samples were analyzed by flow cytometry with a BD FACSCanto I (BD Biosciences, San Jose, CA). A 575 nm phycoerythrin (PE) channel was used as the reporter channel and a 660 nm allophycocyanin (APC) channel was used as the classification channel. A linear mode forward and side scatter were used. Standard samples were first run to generate a standard curve followed by the experimental samples. Sample cytokine concentrations were determined using the Legendplex Data Analysis Software (BioLegend, San Diego, CA).

### RNA Sequencing

In any of the above discussed examples, tissue from system 700 was subject to similar RNA sequencing. For example, tumor bearing peritoneum samples were collected on day 0 and day 4 to assess for conservation of RNA expression. Selection of gene panels was made from commercially available methods that had been previously empirically validated. RNA was extracted from formalin fixed, paraffin embedded samples of tumor and 10 ng of RNA from each sample was used.

### Tumor Imaging

In any of the above discussed examples, tissue from system 700 was subject to similar tumor imaging techniques. For example, for live imaging, sample platforms were removed from the system 700 at indicated time points and transferred to a 24 well plate containing 1 ml perfusate supplemented with 0.5 μg fluorophore-conjugated antibodies (CD45; CD44; CD117) and incubated for 3 hours at 37° C. Sample platforms 300 were first secured in the first member 806 of imaging platform holder 800. The first member 806 was then screwed into second member 814 that fit on dish 820 with a coverglass 836 on its bottom. Following live imaging, the sample platforms 300 were retrieved from the imaging platform holder 800 and returned to the perfusion circuit to be reimaged at a later timepoint.

Image acquisition was performed on an inverted Leica SP8 setup using multiphoton excitation at 870 nm together with a 25×/0.95 W VISIR lens. For the timeseries, images were collected at 2-minute intervals for indicated time periods. Images were analyzed using ImageJ software. Following live imaging, tissue was preserved in a fixative solution containing 1% paraformaldehyde (diluted 1:4) at 4° C. for 24 hours, washed thoroughly with PBS, and then incubated in a 30% sucrose solution for 3 days. After incubation, specimens were removed from the sample platform 300 and mounted vertically in an embedding frame (HistoMold, 6×8 mm) with the perimeter of the tissue situated perpendicular to the base of the mold. The specimen was frozen with Optimum Cutting Temperature Compound (Tissue-Tek) on dry ice. The tissue was then sliced into 30 μm sections and placed on polarized microslides. The sections were incubated in a blocking solution containing 1% BSA, 0.3% Triton-X 100, and 0.05% NaN₃ for 24 hours. After blocking, samples were then stained in primary antibody solutions for 48 hours, and secondary antibody solutions, if needed, for an additional 24 hours. Once staining was completed, samples were mounted with Fluoromount-G (Invitrogen) and secured with a cover glass. Images were acquired with an inverted Leica SP8 microscope equipped with a white light laser, using a 40× objective lens. Images were then processed with the Imaris software.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Clause 1. An ex vivo tissue analysis method, comprising:
(a) mounting a portion of live tissue resected from a patient on a sample platform;
(b) using the sample platform, positioning the resected tissue portion within a perfusion chamber; and
(c) flowing perfusate through the perfusion chamber and into contact with the resected tissue portion such that diffusion of oxygen occurs between the perfusate and the resected tissue portion,
wherein during the flowing, the resected tissue portion maintains a competent immune system.

Clause 2. The ex vivo tissue analysis method of any example herein, particularly Clause 1, wherein the flowing the perfusate includes circulating the perfusate from an outlet of the perfusion chamber to an inlet of the perfusion chamber.

Clause 3. The ex vivo tissue analysis method of any example herein, particularly any one of Clauses 1-2, wherein during the flowing, perfusate within the perfusion chamber is agitated or mixed by passive structures within the perfusion chamber, by active structures within the perfusion chamber, or both.

Clause 4. The ex vivo tissue analysis method of any example herein, particularly Clause 3, wherein the active structures include a stirrer bar that is rotated by an external magnetic field.

Clause 5. The ex vivo tissue analysis method of any example herein, particularly any one of Clauses 3-4, wherein the passive structures include one or more baffles within the perfusion chamber, configuration of inlet and/or outlet ports within the perfusion chamber, arrangement of inlet and/or outlet ports within the perfusion chamber, orientation of perfusate flow within the perfusion chamber, or any combination of the foregoing.

Clause 6. The ex vivo tissue analysis method of any example herein, particularly any one of Clauses 1-5, wherein the resected live tissue comprises part of a solid tumor.

Clause 7. The ex vivo tissue analysis method of any example herein, particularly Clause 6, wherein the solid tumor is a metastasis of a primary cancerous tumor.

Clause 8. The ex vivo tissue analysis method of any example herein, particularly any one of Clauses 6-7, wherein the resected live tissue comprises a heterogenous human tumor microenvironment (TME) including 3-D tissue structure, stromal components, and immune populations.

Clause 9. The ex vivo tissue analysis method of any example herein, particularly any one of Clauses 1-8, wherein the resected live tissue comprises a surface portion of a mesothelium of the patient.

Clause 10. The ex vivo tissue analysis method of any example herein, particularly any one of Clauses 1-9, wherein the perfusate comprises blood plasma or culture medium combined with blood plasma.

Clause 11. The ex vivo tissue analysis method of any example herein, particularly Clause 10, wherein the blood plasma is from the patient (e.g., autologous plasma).

Clause 12. The ex vivo tissue analysis method of any example herein, particularly any one of Clauses 1-11, further comprising:
isolating peripheral blood mononuclear cells (PBMCs) from blood of the patient; and
introducing the isolated PBMCs to the resected tissue portion via the flowing the perfusate.

Clause 13. The ex vivo tissue analysis method of any example herein, particularly any one of Clauses 1-12, wherein, during the flowing, the perfusate is periodically or continuously infused with a drug, a hormone, water, or any combination of the foregoing.

Clause 14. The ex vivo tissue analysis method of any example herein, particularly Clause 13, wherein the hormone comprises insulin.

Clause 15. The ex vivo tissue analysis method of any example herein, particularly any one of Clauses 1-14, further comprising, during or before the flowing, introducing a drug for the resected tissue portion into the perfusate.

Clause 16. The ex vivo tissue analysis method of any example herein, particularly any one of Clauses 1-15, further comprising:
(d) after (c), removing the sample platform with resected tissue portion from the perfusion chamber;
(e) after (d), exposing the resected tissue portion to a cancer treatment without removing the resected tissue portion from the sample platform;
(f) after (e), returning the sample platform to the perfusion chamber; and
(g) after (f), repeating (c).

Clause 17. The ex vivo tissue analysis method of any example herein, particularly Clause 16, wherein (e) comprises immersing a first surface of the resected tissue portion in a drug (e.g., chemotherapeutic agent) for a predetermined period of time, the first surface being exposed from the sample platform.

Clause 18. The ex vivo tissue analysis method of any example herein, particularly any one of Clauses 1-17, further comprising introducing oxygen to the perfusate by:
an oxygenator connected inline with a flow circuit that returns perfusate exiting from an outlet of the perfusion chamber to an inlet of the perfusion chamber;
an oxygen-generating biomaterial disposed within the perfusate;
a gas mixer that combines oxygen and carbon dioxide into a single flow for dissolution in the perfusate; or
any combination of the foregoing.

Clause 19. The ex vivo tissue analysis method of any example herein, particularly any one of Clauses 1-18, further comprising introducing carbon dioxide to the perfusate so as to change or maintain a pH of the perfusate during the flowing.

Clause 20. The ex vivo tissue analysis method of any example herein, particularly any one of Clauses 1-19, wherein the mounting comprises securing a periphery of the resected tissue portion to a circumferential portion of the sample platform.

Clause 21. The ex vivo tissue analysis method of any example herein, particularly Clause 20, wherein the securing is by way of a suture.

Clause 22. The ex vivo tissue analysis method of any example herein, particularly Clause 20, wherein the securing is by way of an annular flexible member that applies a radial compressive force to the periphery of the resected tissue portion.

Clause 23. The ex vivo tissue analysis method of any example herein, particularly Clause 22, wherein the securing includes:
disposing the annular flexible member on an outer circumferential surface of an applicator, the applicator having a truncated cone shape with a circumference at a first axial end being less than that at a second axial end;
disposing the resected tissue portion on a tissue mount section at a first end of the sample platform such that at least part of the resected tissue portion overhangs the tissue mount section,
disposing the second axial end of the applicator proximal to the first end of the sample platform; and
advancing the annular flexible member along the outer circumferential surface toward and over the second axial end of the applicator, such that the annular flexible member comes into contact with the overhanging part of the resected tissue portion.

Clause 24. The ex vivo tissue analysis method of any example herein, particularly Clause 23, wherein the annular flexible member comprises a rubber band, and the advancing comprises rolling or sliding the rubber band along the outer circumferential surface of the applicator.

Clause 25. The ex vivo tissue analysis method of any example herein, particularly any one of Clauses 1-24, wherein:
the mounted tissue portion has a first side facing a tissue mount section of the sample platform and a second side facing away from tissue mount section, and
the tissue mount section has an opening therein that exposes the second side of the mounted tissue portion such that both of the first and second sides are in contact with perfusate in the perfusion chamber.

Clause 26. The ex vivo tissue analysis method of any example herein, particularly any one of Clauses 1-25, further comprising resecting the portion of live tissue from the patient.

Clause 27. The ex vivo tissue analysis method of any example herein, particularly any one of Clauses 1-26, further comprising, after the flowing the perfusate:
removing the sample platform from the perfusion chamber;
positioning the sample platform with respect to a stage of a microscope for imaging of the resected tissue portion; and
imaging the mounted portion of the resected tissue using the microscope.

Clause 28. The ex vivo tissue analysis method of any example herein, particularly Clause 27, further comprising, after the imaging, returning the resected tissue portion back to the perfusion chamber, and continuing the flowing of the perfusate through the perfusion chamber.

Clause 29. The ex vivo tissue analysis method of any example herein, particularly any one of Clauses 27-28, wherein the positioning the sample platform comprises:
mounting the sample platform to an imaging platform holder; and
adjusting an axial position of the imaging platform holder such that the mounted portion of the resected tissue is at a focal plane of the microscope.

Clause 30. The ex vivo tissue analysis method of any example herein, particularly Clause 29, wherein the adjusting an axial position of the imaging platform holder comprises rotating a threaded male shaft within a threaded female recess.

Clause 31. The ex vivo tissue analysis method of any example herein, particularly any one of Clauses 1-30, further comprising:
removing the sample platform from the perfusion chamber;
providing a ring gasket around a portion of the sample platform, the ring gasket forming a well adjacent to the resected tissue portion; and
filling at least part of the well with blood plasma, culture medium, or both.

Clause 32. The ex vivo tissue analysis method of any example herein, particularly Clause 31, wherein the ring gasket is formed of an oxygen-generating polymer, and the providing the ring gasket and the filling at least part of the well are effective to create an oxygen microenvironment for the resected tissue portion while outside of the perfusion chamber.

Clause 33. A system for ex vivo tissue analysis, comprising:
a perfusion chamber having an inlet, an outlet, and internal volume between the inlet and outlet; and
a sample platform having a tissue mount section and a chamber mount section coupled to the tissue mount section, the tissue mount section being constructed for mounting of a resected tissue portion thereon, the chamber mount section being constructed to releasably support the sample platform with respect to the perfusion chamber such that the resected tissue portion is positioned within the internal volume of the perfusion chamber,
wherein the tissue mount section of the sample platform has an opening that exposes a backside of the mounted resected tissue portion, such that both a frontside and a backside of the mounted resected tissue portion are exposed to perfusate within the perfusion chamber.

Clause 34. The system for ex vivo tissue analysis of any example herein, particularly Clause 33, wherein:
the tissue mount section comprises an annular platform having a first outer diameter,
the chamber mount section comprises a circular base having a second outer diameter greater than the first outer diameter, and
the sample platform comprises one or more arms that extend between and connect the annular platform to the circular base.

Clause 35. The system for ex vivo tissue analysis of any example herein, particularly Clause 34, wherein two arms extend between and connect the annular platform to the circular base, the arms being on opposite sides of the opening in the annular platform from each other, and each arm has one or more through-holes or openings in a portion proximal to the annular platform.

Clause 36. The system for ex vivo tissue analysis of any example herein, particularly any one of Clauses 34-35, wherein the annular platform includes a circumferential groove configured to receive an attachment member for securing a periphery of the mounted resected tissue portion to the annular platform.

Clause 37. The system for ex vivo tissue analysis of any example herein, particularly any one of Clauses 34-36, wherein:
the perfusion chamber includes a top surface with an opening having a third diameter, which is greater than the first outer diameter and less than the second outer diameter, and
the sample platform is constructed such that chamber mount section is supported on the top surface of the perfusion chamber while the tissue mount section extends through opening via the one or more arms to suspend the mounted tissue portion within the internal volume of the perfusion chamber.

Clause 38. The system for ex vivo tissue analysis of any example herein, particularly any one of Clauses 33-37, wherein the inlet of the perfusion chamber is at a height with respect to a bottom surface of the perfusion chamber that is different from that of the outlet of the perfusion chamber.

Clause 39. The system for ex vivo tissue analysis of any example herein, particularly Clause 38, wherein the inlet is higher than the outlet.

Clause 40. The system for ex vivo tissue analysis of any example herein, particularly any one of Clauses 33-39, further comprising an oxygen-generating biomaterial (OGB) disposed within the perfusion chamber, the OGB being constructed to release oxygen into perfusate within the internal volume of the perfusion chamber.

Clause 41. The system for ex vivo tissue analysis of any example herein, particularly any one of Clauses 33-40, further comprising a pump that moves perfusate through the internal volume of the perfusion chamber via flow into the inlet and out of the outlet.

Clause 42. The system for ex vivo tissue analysis of any example herein, particularly Clause 41, further comprising a fluid circuit with fluid conduits connecting the outlet of the perfusion chamber to the inlet of the perfusion chamber, wherein the pump is constructed to circulate perfusate through the fluid conduits and to flow the perfusate through the internal volume of the perfusion chamber.

Clause 43. The system for ex vivo tissue analysis of any example herein, particularly Clause 33-42, further comprising:
a gas exchanger constructed to provide oxygen to the perfusate, removing carbon dioxide from the perfusate, or both;
a gas mixer for mixing oxygen and carbon dioxide in a single gas flow for dissolution in the perfusate;
a pressure control valve for venting carbon dioxide removed from the perfusate; or any combination of the foregoing.

Clause 44. The system for ex vivo tissue analysis of any example herein, particularly Clause 43, wherein the gas exchanger comprises an oxygenator.

Clause 45. The system for ex vivo tissue analysis of any example herein, particularly any one of Clauses 42-44, wherein the fluid circuit comprises a sampling port or valve, through which a portion or all of the perfusate is removed from the fluid circuit.

Clause 46. The system for ex vivo tissue analysis of any example herein, particularly any one of Clauses 42-45, wherein the fluid circuit comprises an infusion port or valve, through which a drug, a hormone, cells, fluid, or any combination of the foregoing is introduced into the fluid conduits.

Clause 47. The system for ex vivo tissue analysis of any example herein, particularly any one of Clauses 33-46, further comprising an incubator constructed to maintain a predetermined temperature for components therein, at least the perfusion chamber being disposed within the incubator.

Clause 48. The system for ex vivo tissue analysis of any example herein, particularly any one of Clauses 33-47, further comprising a passive structure that agitates or mixes perfusate within the perfusion chamber, an active structure that agitates or mixes perfusate within the perfusion chamber, or any combination of the foregoing.

Clause 49. The system for ex vivo tissue analysis of any example herein, particularly Clause 48, wherein the active structure comprises a stirrer bar within the internal volume of the perfusion chamber that is rotated by an externally applied magnetic field.

Clause 50. The system for ex vivo tissue analysis of any example herein, particularly any one of Clauses 48-49, wherein the passive structure comprises baffles within the perfusion chamber, arrangement of inlet and/or outlet ports within the perfusion chamber, orientation of perfusate flow within the perfusion chamber, or any combination of the foregoing.

Clause 51. The system for ex vivo tissue analysis of any example herein, particularly any one of Clauses 33-50, further comprising a controller coupled to and controlling operation of the pump, the gas exchanger, the CO₂ pressure control valve, the sampling port or valve, the infusion port or valve, the incubator, or any combination of the foregoing.

Clause 52. The system for ex vivo tissue analysis of any example herein, particularly Clause 51, wherein the controller comprises:
one or more processors; and
computer-readable storage media storing computer-readable instructions that, when executed by the one or more processors, cause the one or more processors to control the system to perform the method of any of Clauses 1-32.

Clause 53. The system for ex vivo tissue analysis of any example herein, particularly any one of Clauses 33-52, further comprising an imaging platform holder constructed to receive the sample platform therein for microscopic imaging of the mounted tissue portion.

Clause 54. The system for ex vivo tissue analysis of any example herein, particularly Clause 53, wherein the imaging platform holder comprises:
a first member having a threaded shaft, a through-hole extending through a center of the threaded shaft, and a top surface with a pair of securing arms on opposite sides of the through-hole;
a second member having a through-hole with threads complementary to that of the threaded shaft of the first member and constructed to receive the threaded shaft therein; and
a locking member constructed to be disposed between the chamber mount section of the sample platform and the securing arms to releasably couple the sample platform to the first member,
wherein a diameter of the through-hole of the first member is greater than an outer diameter of the tissue mount section and less than an outer diameter of the chamber mount section.

### CONCLUSION

For components of the disclosed systems that come into contact with the tissue sample (either directly or indirectly via fluid), such components can be made of any suitable biocompatible material. If reusability of components is desired, the components can be made of a biocompatible material that is also autoclavable for sterilization. For example, embodiments of the disclosed sample holder can be formed of a composite resin (e.g., a dental resin including bisphenol A-glycidyl methacrylate), medical grade stainless steel, glass, or a polymer (e.g., a fluoropolymer such as polytetrafluoroethylene (PTFE)).

Although some of the embodiments described above refer to “imaging,” the production of an actual image is not strictly necessary. Indeed, the mentions of “imaging” are intended to include the acquisition of data where an image may not be produced. Accordingly, the use of the term “imaging” herein should not be understood as limiting.

Any of the features illustrated or described with respect to FIGS. 1A-12D, Examples 1-5, and Clauses 1-54 can be combined with any other of FIGS. 1A-12D, Examples 1-5, and Clauses 1-54 to provide systems, methods, devices, and embodiments not otherwise illustrated or specifically described herein. For example, cooperating features to align and/or retain components, similar to that illustrated in FIG. 1C, can be used in engagement between the perfusion chamber 600 and the environmental control apparatus (e.g., incubator in FIG. 7A), engagement between the lid 608 and bottom dish 616 of the perfusion chamber 600, and/or engagement between the sample platform and an exposure platform holder (e.g., FIGS. 1F-1G). In another example, the ring gasket of FIG. 1E can be applied to any of the disclosed uses of sample platform external to the perfusion chamber, such as discussed with respect to FIGS. 1D, 1F, and 1G. Other combinations and variations are also possible according to one or more contemplated embodiments. Indeed, all features described herein are independent of one another and, except where structurally impossible, can be used in combination with any other feature described herein.

In view of the many possible embodiments to which the principles of the disclosed technology may be applied, it should be recognized that the illustrated embodiments are only preferred examples and should not be taken as limiting the scope of the disclosed technology. Rather, the scope is defined by the following claims. We therefore claim all that comes within the scope and spirit of these claims.

1. An ex vivo tissue analysis method, comprising:
(a) mounting a portion of live tissue resected from a patient on a sample platform;
(b) using the sample platform, positioning the resected tissue portion within a perfusion chamber; and
(c) flowing perfusate through the perfusion chamber and into contact with the resected tissue portion such that diffusion of oxygen occurs between the perfusate and the resected tissue portion,
wherein during the flowing, the resected tissue portion maintains a competent immune system.

2. The ex vivo tissue analysis method of claim 1, wherein the flowing the perfusate includes circulating the perfusate from an outlet of the perfusion chamber to an inlet of the perfusion chamber.

3. The ex vivo tissue analysis method of claim 1, wherein during the flowing, perfusate within the perfusion chamber is agitated or mixed by passive structures within the perfusion chamber, by active structures within the perfusion chamber, or by both.

4. The ex vivo tissue analysis method of claim 3, wherein the passive structures include baffles within the perfusion chamber, configuration and/or arrangement of inlet and/or outlet ports within the perfusion chamber, and/or orientation of perfusate flow within the perfusion chamber.

5. The ex vivo tissue analysis method of claim 1, wherein the resected live tissue comprises part of a solid tumor.

6. The ex vivo tissue analysis method of claim 5, wherein the solid tumor is a metastasis of a primary cancerous tumor.

7. The ex vivo tissue analysis method of claim 6, wherein the resected live tissue comprises a heterogenous human tumor microenvironment (TME) including 3-D tissue structure, stromal components, and immune populations.

8. The ex vivo tissue analysis method of claim 1, wherein the resected live tissue comprises a surface portion of a mesothelium of the patient.

9. The ex vivo tissue analysis method of claim 1, wherein the perfusate comprises blood plasma or culture medium combined with blood plasma.

10. The ex vivo tissue analysis method of claim 9, wherein the blood plasma is from the patient.

11. The ex vivo tissue analysis method of claim 1, further comprising:
isolating peripheral blood mononuclear cells (PBMCs) from blood of the patient; and
introducing the isolated PBMCs to the resected tissue portion via the flowing the perfusate.

12. The ex vivo tissue analysis method of claim 1, wherein, during the flowing, the perfusate is periodically or continuously infused with a drug, a hormone, water, or any combination of the foregoing.

13. The ex vivo tissue analysis method of claim 12, wherein the hormone comprises insulin.

14. The ex vivo tissue analysis method of claim 1, further comprising, during or before the flowing, introducing a drug for the resected tissue portion into the perfusate.

15. The ex vivo tissue analysis method of claim 1, further comprising:
(d) after (c), removing the sample platform with resected tissue portion from the perfusion chamber;
(e) after (d), exposing the resected tissue portion to a cancer treatment without removing the resected tissue portion from the sample platform;
(f) after (e), returning the sample platform to the perfusion chamber; and
(g) after (f), repeating (c).

16. The ex vivo tissue analysis method of claim 1, further comprising introducing oxygen to the perfusate by:
an oxygenator connected inline with a flow circuit that returns perfusate exiting from an outlet of the perfusion chamber to an inlet of the perfusion chamber;
an oxygen-generating biomaterial disposed within the perfusate;
a gas mixer that combines oxygen and carbon dioxide into a single flow for dissolution in the perfusate; or
any combination of the foregoing.

17. The ex vivo tissue analysis method of claim 1, further comprising:
introducing carbon dioxide to the perfusate so as to change or maintain a pH of the perfusate during the flowing.

18. The ex vivo tissue analysis method of claim 1, wherein the mounting comprises securing a periphery of the resected tissue portion to a circumferential portion of the sample platform.

19. The ex vivo tissue analysis method of claim 18, wherein the securing is by way of a suture.

20. The ex vivo tissue analysis method of claim 18, wherein the securing is by way of an annular flexible member that applies a radial compressive force to the periphery of the resected tissue portion.

21. The ex vivo tissue analysis method of claim 20, wherein the securing includes:
disposing the annular flexible member on an outer circumferential surface of an applicator, the applicator having a truncated cone shape with a circumference at a first axial end being less than that at a second axial end;
disposing the resected tissue portion on a tissue mount section at a first end of the sample platform such that at least part of the resected tissue portion overhangs the tissue mount section,
disposing the second axial end of the applicator proximal to the first end of the sample platform; and
advancing the annular flexible member along the outer circumferential surface toward and over the second axial end of the applicator, such that the annular flexible member comes into contact with the overhanging part of the resected tissue portion.

22. The ex vivo tissue analysis method of claim 21, wherein the annular flexible member comprises a rubber band, and the advancing comprises rolling or sliding the rubber band along the outer circumferential surface of the applicator.

23. The ex vivo tissue analysis method of claim 1, wherein:
the mounted tissue portion has a first side facing a tissue mount section of the sample platform and a second side facing away from tissue mount section, and
the tissue mount section has an opening therein that exposes the second side of the mounted tissue portion such that both of the first and second sides are in contact with perfusate in the perfusion chamber.

24. The ex vivo tissue analysis method of claim 1, further comprising resecting the portion of live tissue from the patient.

25. The ex vivo tissue analysis method of claim 1, further comprising, after the flowing the perfusate:
removing the sample platform from the perfusion chamber;
positioning the sample platform with respect to a stage of a microscope for imaging of the resected tissue portion; and
imaging the mounted portion of the resected tissue using the microscope.

26. The ex vivo tissue analysis method of claim 25, further comprising, after the imaging, returning the resected tissue portion back to the perfusion chamber, and continuing the flowing of the perfusate through the perfusion chamber.

27. The ex vivo tissue analysis method of claim 25, wherein the positioning the sample platform comprises:
mounting the sample platform to an imaging platform holder; and
adjusting an axial position of the imaging platform holder such that the mounted portion of the resected tissue is at a focal plane of the microscope.

28. The ex vivo tissue analysis method of claim 27, wherein the adjusting an axial position of the imaging platform holder comprises rotating a threaded male shaft within a threaded female recess.

29. The ex vivo tissue analysis method of claim 1, further comprising:
removing the sample platform from the perfusion chamber;
providing a ring gasket around a portion of the sample platform, the ring gasket forming a well adjacent to the resected tissue portion; and
filling at least part of the well with blood plasma, culture medium, or both.

30. The ex vivo tissue analysis method of claim 29, wherein the ring gasket is formed of an oxygen-generating polymer, and the providing the ring gasket and the filling at least part of the well are effective to create an oxygen microenvironment for the resected tissue portion while outside of the perfusion chamber.

31. A system for ex vivo tissue analysis, comprising:
a perfusion chamber having an inlet, an outlet, and internal volume between the inlet and outlet; and
a sample platform having a tissue mount section and a chamber mount section coupled to the tissue mount section, the tissue mount section being constructed for mounting of a resected tissue portion thereon, the chamber mount section being constructed to releasably support the sample platform with respect to the perfusion chamber such that the resected tissue portion is positioned within the internal volume of the perfusion chamber,
wherein the tissue mount section of the sample platform has an opening that exposes a backside of the mounted resected tissue portion, such that both a frontside and a backside of the mounted resected tissue portion are exposed to perfusate within the perfusion chamber.

32. The system for ex vivo tissue analysis of claim 31, wherein:
the tissue mount section comprises an annular platform having a first outer diameter, the chamber mount section comprises a circular base having a second outer diameter greater than the first outer diameter, and
the sample platform comprises one or more arms that extend between and connect the annular platform to the circular base.

33. The system for ex vivo tissue analysis of claim 32, wherein two arms extend between and connect the annular platform to the circular base, the arms being on opposite sides of the opening in the annular platform from each other, and each arm has one or more through-holes or openings in a portion proximal to the annular platform.

34. The system for ex vivo tissue analysis of claim 32, wherein the annular platform includes a circumferential groove configured to receive an attachment member for securing a periphery of the mounted resected tissue portion to the annular platform.

35. The system for ex vivo tissue analysis of claim 32, wherein:
the perfusion chamber includes a top surface with an opening having a third diameter, which is greater than the first outer diameter and less than the second outer diameter, and
the sample platform is constructed such that chamber mount section is supported on the top surface of the perfusion chamber while the tissue mount section extends through opening via the one or more arms to suspend the mounted tissue portion within the internal volume of the perfusion chamber.

36. The system for ex vivo tissue analysis of claim 31, wherein the inlet of the perfusion chamber is at a height with respect to a bottom surface of the perfusion chamber that is different from that of the outlet of the perfusion chamber.

37. The system for ex vivo tissue analysis of claim 36, wherein the inlet is higher than the outlet.

38. The system for ex vivo tissue analysis of claim 31, further comprising an oxygen-generating biomaterial (OGB) disposed within the perfusion chamber, the OGB being constructed to release oxygen into perfusate within the internal volume of the perfusion chamber.

39. The system for ex vivo tissue analysis of claim 31, further comprising a pump that moves perfusate through the internal volume of the perfusion chamber via flow into the inlet and out of the outlet.

40. The system for ex vivo tissue analysis of claim 39, further comprising a fluid circuit with fluid conduits connecting the outlet of the perfusion chamber to the inlet of the perfusion chamber, wherein the pump is constructed to circulate perfusate through the fluid conduits and to flow the perfusate through the internal volume of the perfusion chamber.

41. The system for ex vivo tissue analysis of claim 40, further comprising:
a gas exchanger constructed to provide oxygen to the perfusate, removing carbon dioxide from the perfusate, or both;
a gas mixer for mixing oxygen and carbon dioxide in a single gas flow for dissolution in the perfusate;
a pressure control valve for venting carbon dioxide removed from the perfusate; or
any combination of the foregoing.

42. The system for ex vivo tissue analysis of claim 41, wherein the gas exchanger comprises an oxygenator.

43. The system for ex vivo tissue analysis of claim 40, wherein the fluid circuit comprises:
a sampling port or valve, through which a portion or all of the perfusate is removed from the fluid circuit;
an infusion port or valve, through which a drug, a hormone, cells, and/or fluid is introduced into the fluid conduits; or
any combination of the foregoing.

44. The system for ex vivo tissue analysis of claim 31, further comprising an incubator constructed to maintain a predetermined temperature for components therein, at least the perfusion chamber being disposed within the incubator.

45. The system for ex vivo tissue analysis of claim 31, further comprising a passive structure that agitates or mixes perfusate within the perfusion chamber, an active structure that agitates or mixes perfusate within the perfusion chamber, or any combination of the foregoing.

46. The system for ex vivo tissue analysis of claim 45, wherein the passive structure comprises baffles within the perfusion chamber, arrangement of inlet and/or outlet ports within the perfusion chamber, orientation of perfusate flow within the perfusion chamber, or any combination of the foregoing.

47. The system for ex vivo tissue analysis of claim 31, further comprising an imaging platform holder constructed to receive the sample platform therein for microscopic imaging of the mounted tissue portion.

48. The system for ex vivo tissue analysis of claim 47, wherein the imaging platform holder comprises:
a first member having a threaded shaft, a through-hole extending through a center of the threaded shaft, and a top surface with a pair of securing arms on opposite sides of the through-hole;
a second member having a through-hole with threads complementary to that of the threaded shaft of the first member and constructed to receive the threaded shaft therein; and
a locking member constructed to be disposed between the chamber mount section of the sample platform and the securing arms to releasably couple the sample platform to the first member,
wherein a diameter of the through-hole of the first member is greater than an outer diameter of the tissue mount section and less than an outer diameter of the chamber mount section.
